(19) United States
(12) Patent Application Publication
(10) Pub. No.: US 2025/0273327 A1
(43) Pub. Date: Aug. 28, 2025

(54) SYSTEMS AND METHODS FOR DYNAMIC INFECTION MONITORING AND TRACKING

(71) Applicant: HACKENSACK MERIDIAN HEALTH, INC., Edison, NJ (US)

(72) Inventors: Ariel Aptekmann, Buenos Aires (AR); Barry Kreiswirth, Edison, NJ (US)

(73) Assignee: HACKENSACK MERIDIAN HEALTH, INC., Edison, NJ (US)

(21) Appl. No.: 19/066,799

(22) Filed: Feb. 28, 2025

Related U.S. Application Data
(60) Provisional application No. 63/559,131, filed on Feb. 28, 2024.

(51) Int. Cl. G16H 40/20; G16H 10/60

(52) U.S. Cl. CPC G16H 40/20; G16H 10/60

(57) ABSTRACT

Embodiments described herein include systems and methods for dynamic infection monitoring and tracking for healthcare facilities and healthcare systems. Some embodiments obtain information from electronic medical record systems, identify potentially related infections, identify patient contacts with healthcare workers and instrument based on contacts documents in electronic health records and based on geographic and time overlap between patients and healthcare workers. Some embodiments determine likely infection propagation paths based on the potentially related infections and patient contacts. Some embodiments generate a dynamic interactive user interface enabling a user to view and interact with visualizations of infection monitoring and tracking results.

### BACKGROUND

A major problem in hospitals and health care facilities today is the prevalence of hospital-acquired infections. Infections picked up in institutions are referred to as “nosocomial” infections.

Unfortunately, many bacteria develop resistance to the drugs that are used to fight them. As a result of the high levels of antibiotic usage, hospitals provide a selective environment to aid in the spread of drug resistant bacteria. Bacterial infections get worse over time because the bacteria become more resistant to the drugs used to treat them. The more resistant the bacteria get, the harder they are to eradicate and the more they linger in the hospital environment.

Health care facilities today live with a baseline level of nosocomial infections among patients. Some hospitals do not take active steps to control nosocomial infections until a significant number of patients acquire infections within a short period of time. Unfortunately, by the time that the hospital realizes that it has an outbreak problem, the outbreak may have already been underway for weeks to months. Thus, the hospital will already have expended significant resources to fighting the spread of infection, and will have to expend additional resources to eradicate the infection from the hospital.

When the infection has already become rampant, the hospital may try to combat the outbreak by locating the source of the infection. The source could be a patient in the hospital, a health care worker, an animal, a contaminated object, such as a bronchoscope, a prosthetic device, the plumbing in a dialysis machine, or a myriad of other locations. It is thus very important that the hospital be able to locate the source of the infection.

When a patient acquires an infection in a hospital, typically an isolate of the bacteria will be taken from the patient and sent to a laboratory. The laboratory performs phenotypic tests to determine the species of the bacteria and its antibiotic susceptibility profile, which provides the physician a guide to the proper antibiotic therapy. Phenotypic tests examine the physical and biological properties of the cell, as opposed to genotypic tests, which evaluate the DNA content of the cell's genes.

The hospital can attempt to locate the source of infection by determining the path of transmission of the infection. The hospital can potentially determine the path of transmission by subspeciating the bacteria. One way to subspeciate bacteria is to analyze the bacteria's DNA. This is referred to as “molecular” typing, or genotyping. Over time, a bacteria's DNA mutates, producing changes in the bacteria's DNA. Two isolates of bacteria taken from two different patients may appear to have identical physical properties or “phenotypic” characteristics. However, a closer examination of the bacterial DNA might reveal subtle differences that demonstrate that the two isolates are actually different subspecies or clonal types. As an example, genotypic tests compare the DNA of a given gene from two or more organism, whereas phenotypic tests compare the expression of those genes.

If the hospital determines that many patients are acquiring infections of the same species, then the hospital may suspect that it has an outbreak problem. In some cases drug susceptibility testing will determine that microbial strains are different and that an outbreak has not occurred. Unfortunately, many outbreaks are cause by multidrug resistant organisms, which cannot be distinguished solely based on drug susceptibility results. In these cases, sub-speciation data is necessary to distinguish microbial strain types. Molecular typing is one effective way to subspeciate these strains. Rarely do hospitals perform molecular typing to subspeciate bacteria (i.e. a DNA analysis) because they lack the tools and expertise. In addition, in the age of HMO care, preventive typing does not constitute direct patient care; it is infection control. However, in the long run, the hospital pays increased costs because patient stays are longer as a direct result of nosocomial infections.

Reducing the incidence of hospital-acquired infections would improve patient health and outcomes and may reduce the average length of stay for a patient in a hospital. In many areas of the United States, there are insufficient healthcare resources (e.g., hospital beds) for the current needs. Reducing the incidence of hospital acquired infections and reducing the average length of stay of a patient in a hospital may reduce the strain on limited resources and may enable additional people to access needed health care.

Currently, most hospital visits in the United States are paid for by Health Maintenance Organizations (HMOs). Extended patient stays caused by complications unrelated to the intended procedure, such as hospital-acquired infections, are often not covered by the HMO's. These extra costs are paid for by the hospitals. Hospital acquired infections equate to extended patient stays and extended patient treatment. Reducing hospital infection rates would reduce the length of patient stays, and thus save a significant amount of money for hospitals, HMO's and ultimately patients as well as reduce strains on often overcrowded hospitals.

What is also needed is a system that responds to an outbreak at a very early stage rather than beginning weeks or months after an outbreak has already begun.

### SUMMARY

Some embodiments provide methods, system and non-transitory computer readable media for infection tracking and monitoring in a healthcare facility or a healthcare system.

In accordance with one aspect, a method for infection tracking and monitoring in a healthcare facility or a healthcare system is provided. The method includes: accessing or receiving current information from electronic medical records or an electronic medical records system and a laboratory information system, the current information including: patient data, healthcare worker data, instrument data, and infection data; the patient data including, for each patient identified as having an infection: an identifier for the patient, demographic information, medical history, and admission information where admitted; the healthcare worker data including, for each healthcare worker: an identifier for the healthcare worker, a role or position of the healthcare worker; the instrument data including for each instrument: an identifier for the instrument and record information about the usage of the instrument; and the infection data including: cases of infections among patients including, for each case: type of infection, and date of diagnosis. The method also includes storing the current information in an infection monitoring and tracking (IMaT) database. The method also includes accessing, receiving, or generating additional current information including updated patient data, updated healthcare worker data, updated instrument data, and updated infection data periodically or on demand and storing the additional current information in the IMaT database, the current information and the additional current information identified herein as collected information. The method also includes determining interaction data for each patient from the collected information based on documented interactions between the patient and one or more healthcare workers, based on documented uses of one or more instruments on the patient, and based on spatial location, date, and time overlap between the patient and the one or more healthcare workers and storing the determined interaction data in the IMaT database. The method also includes identifying potentially related infections based, at least in part, on the infection data. The method also includes determining one or more likely infection propagation paths based, at least in part, on the identified potentially related infections and the interaction data. The method also includes generating a dynamic interactive user interface enabling a user to view and interact with visualizations of infection monitoring and tracking results and/or generating a report on infection status including infection monitoring and tracking results where the infection monitoring and tracking results include the determined one or more likely infection propagation paths.

In some embodiments, wherein the infection data further includes microbial strain characterization of the infection for at least some of the infections. In some embodiments, the microbial strain characterization of infection data includes antibiogram data, genome sequencing data, or both for at least some of the infections.

In some embodiments, the method also includes generating a spatially explicit infection propagation view depicting spatial relationships between infected patients and a structure of at least a portion of a healthcare facility for display in the dynamic interactive user interface based on the determined likely propagation paths.

In some embodiments, the spatially explicit infection propagation view includes graphical representations of contacts between individual infected patients identified as having a potentially related infection. In some embodiments, the dynamic interactive user interface is configured to receive a selection of a graphical representation of contacts between two individual infected patients identified as having a potentially related infection from a user and to display details regarding the contact based on receiving the selection. In some embodiments, the dynamic interactive user interface is configured to receive input from a user regarding at least one parameter for identifying potentially related infections based on the infection data and to display a resulting change to the graphical representations of contacts and/or to the patients identified as having potentially related infections.

In some embodiments, the collected data is preprocessed, normalized, or both prior to being stored in the IMaT database. In some embodiments, the collected data stored in the IMaT database is analyzed to generate enriched data and wherein the enriched data includes at least some of the interaction data.

In some embodiments, the method includes generating one or more spatially implicit models based, at least in part, on the infection data and the patient data. In some embodiments, identifying potentially related infections includes performing a clustering analysis based on one of the spatially implicit models.

In some embodiments, the method includes generating one or more spatially explicit models based, at least in part, on patient data, interaction data, and the identified potentially related infections.

In some embodiments, the likely propagation paths are determined based, at least in part, on the potentially related infections and at least one of the one or more spatially explicit models.

In some embodiments, the method also includes generating or issuing an alert or notification. In some embodiments, the alert or notification includes at least some or all of information on a microbial strain of infection, characteristics of a pathogen for the infection, a location involved, personnel involved, or an instrument involved.

In some embodiments, the alert or notification is a rapid increase alert or notification. In some embodiments, issuance of the rapid increase alert or notification is based on pathogen-specific detection criteria. In some embodiments, the pathogen-specific detection criteria is based on a rate of increase for a specific pathogen being greater than a pathogen-specific threshold based on prior data for the pathogen. In some embodiments, wherein the pathogen-specific threshold is based on prior data for the pathogen. In some embodiments, wherein the pathogen-specific threshold is based on prior data for infections for the pathogen at the healthcare facility or healthcare system.

In some embodiments, the alert or notification is an active propagator alert or notification. In some embodiments, the active propagator alert or notification is issued based on a healthcare worker or an instrument being identified to have been in contact with more than a statistically expected number of patients with potentially related infections in a timeframe.

In some embodiments, the alert or notification is an emerging pathogen alert or notification. In some embodiments, issuance of the emerging pathogen alert or notification is based on one or more of: detection of the appearance of a pathogen having a combination of resistances that has not been previously observed in known pathogens; detection of an infection with an increase in resistance to a drug larger than a determination error for the drug; detection of a non-cultivable pathogen not matching known microbial strains; detection of a pathogen with a genotype not matching previous known data is detected; detection of a pathogen not matching previous known data as determined by genomic sequencing; or detection of a pathogen not matching previous known data as detected by a genomic-based technology.

In some embodiments, the alert or notification includes at least some or all of information on a microbial strain of infection, characteristics of a pathogen for the infection, a location involved, personnel involved, or an instrument involved.

In some embodiments, the method further includes: generating a pathogen propagator's report; or generating a key propagators report.

In some embodiments, the method further includes generating a recommended list of one or more infections for molecular analysis. In some embodiments, the method further includes identifying a one or more infections as a priority for analysis and generating or transmitting an order for a molecular analysis of the one or more infections.

In some embodiments, updated patient data, updated healthcare worker data, updated instrument data, and updated infection data are obtained periodically or on demand at least on a daily basis or more frequently than a daily basis; and determining of interaction data for each patient occurs on a daily basis or more frequently than a daily basis.

In accordance with one aspect, a non-transitory computer readable medium is provided. The non-transitory computer readable medium includes instructions that, when executed by a computing system including one or more processors, cause the computing system to perform any one of the methods described or disclosed herein.

In accordance with one aspect, a system for infection tracking and monitoring for a healthcare facility or a healthcare system. The system includes at least one database including an infection monitoring and tracking (IMaT) database; and one or more processors configured to execute instructions that, when executed by the one or more processors, cause the computing system to perform any of the methods described or disclosed herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. To assist those of skill in the art in making and using the disclosed phantom and associated systems and methods, reference is made to the accompanying figures, wherein:

FIG. 1A is a flow diagram of a method for infection tracking in accordance with some embodiments.

FIG. 1B is a flow diagram of additional steps that may be included in the method for infection tracking of FIG. 1A in accordance with some embodiments.

FIG. 2 schematically depicts an architecture for a system for infection tracking and mapping in accordance with some embodiments.

FIG. 3 schematically depicts information flow, sources, methods and deliverables that may be incorporated into systems for infection tracking and employed by methods for infection tracking in accordance with some embodiments.

FIG. 4 is an example of a dynamic interactive graphical user interface that may be employed in some embodiments.

FIG. 5 is a graph of frequency of different types of infections by infection onset date that may be displayed in a GUI in accordance with some embodiments.

FIG. 6A is graphical depiction of an antibiogram for a first population of infected cells in accordance with some embodiments.

FIG. 6B is graphical depiction of an antibiogram for a second population of infected cells in accordance with some embodiments.

FIG. 6C is a graph with overlaid antibiograms for the first population of infected cells and for the second population of infected cells.

FIG. 7A depicts a map of patient infections that may be displayed on a graphical user interface (GUI), the map of patient infections including geographical or spatial representation of a floor of a healthcare facility including dots indicating patients having a same microbial strain of infection and including an arrow showing a contact between two of the patients in accordance with some embodiments. FIG. 7A depicts day two of a detected infection.

FIG. 7B is a detail of the map of patient infections of FIG. 7A.

FIG. 7C is a map of patient infections corresponding to the same microbial strain of infection for data corresponding to day three of the detected infection depicted in FIGS. 7A and 7B tracing the spread of infection.

FIG. 7D is a map of patient infections corresponding to the same microbial strain of infection for data corresponding to day four of the infection depicted in FIG. 7A and 7B tracing the further spread of infection.

FIG. 7E is a map of patient infections corresponding to the same microbial strain of infection for data corresponding to day five of the infection depicted in FIG. 7A and 7B tracing the further spread of infection.

FIG. 8 depicts another example of a map of patient infections for a different outbreak to be displayed in a GUI with more infected patients and more contacts than those appearing in FIGS. 7A-7E, in accordance with some embodiments.

FIG. 9 schematically depicts a network for implementing some methods or in which or with which some systems may be employed, in accordance with some embodiments.

FIG. 10 schematically depicts a computing system or computing device for implementing some methods or that may be employed in some systems, in accordance with some embodiments.

### DETAILED DESCRIPTION

### Biology Definitions

The term “aerobe” as used herein refers to a microorganism whose growth requires the presence of air or free oxygen.

The term “algae” as used herein refer to a heterogeneous group of eukaryotic, photosynthetic, unicellular and multicellular organisms lacking true tissue differentiation.

The term “allele” as used herein refers to a particular sequence of one or more nucleotides at a chromosomal locus. In a haploid organism, the subject has one allele at every chromosomal locus. In a diploid organism, the subject has two alleles at every chromosomal locus.

The term “anaerobe” as used herein refers to a microorganism able to grow in the absence of air or free oxygen.

The term “antibiogram” as used herein refers to the pattern of sensitivities of a given microorganism towards a range of antibiotics.

The term “antimicrobial susceptibility testing” (“AST”) as used herein refers to a laboratory procedure employed to identify which antimicrobial regimen is specifically effective for the control and prevention of infectious microbial diseases for individual patients. In this context, a susceptible microorganism is inhibited by a concentration of antimicrobial agent that can be attained in blood, which implies that an infection caused by this microorganism may be appropriately treated with the antimicrobial agent. Ed. University of Texas Medical Branch, Galveston, TX (1996): p. 161; Chapter 11, p. 176].

The term “Archaea” as used herein refers to one of the three domains of living cells that are often found living in extreme habitats and that possess unique genetic, biochemical, and physiological characteristics. They represent a primary biological line of evolution related to both bacteria and eukaryotes.

The term “bacteria” as used herein refers to a taxon comprising one of the two fundamentally distinct groups of prokaryotic microorganisms, which lack a nucleus. They are usually single-celled organisms. In size, most bacteria are between 1 and 10 microns in their maximum dimension. The smallest range from <1 micron (e.g. Chlamydia, Francisella, Rickettsia). The basic shapes of bacteria are bacillus (rod), coccus, and spirillum. Generally, the shape of a bacterium is determined primarily by its species. Cells may occur singly or in pairs, chains or clusters. Many types of bacteria are motile. According to species a bacterial cell may have certain appendages, e.g., fimbriae, flagellum, pili, capsule. Some species form exospores;

some form endospores. Although bacterial cells lack the sophisticated physical compartmentalization of eukaryotic cells, they exhibit a functional compartmentalization. The bacterial genome commonly consists of covalently-closed circular DNA; in some bacteria (e.g., Borrelia burgdorferi and species of Streptomyces), the DNA is linear. The number of chromosomes per cell depends e.g., on species and on the growth rate. In many bacteria the genome is supplemented by one or more plasmids; bacterial plasmids are commonly circular, but some are linear. In most species there is a characteristic type of cell wall; the mycoplasmas are atypical in being wall-less. Reproduction occurs asexually, usually by binary fission but sometimes by budding or ternary fission. Despite the lack of sexual reproduction, gene transfer between bacteria can occur by conjugation, conjugative transposition, transduction or transformation. Examples include mycobacteria, Chlamydiae, mycoplasmas, and Rickettsiae.

The terms “bacterial resistance”, “resistant organism”, and “resistance” are used interchangeably to indicate that an infection caused by a resistant organism cannot be successfully treated with a tested antimicrobial agent. The basic mechanisms by which a microorganism can resist an antibacterial agent include (i) to alter the receptor for the agent; (ii) to decrease the amount that reaches the receptor by altering entry or increasing removal of the antibacterial agent; (iii) to destroy or inactivate the agent; (iv) to develop resistant metabolic pathways; (v) to possess resistance-conferring plasmids; (vi) to possess resistance-conferring transposons that can insert into plasmids and also into the bacterial chromosome. Bacteria can possess one or all of these mechanisms simultaneously. [Medical Microbiology, 4th Ed., Samuel Barron,

The term “binary fission” as used herein refers to a process in which two similarly sized and shaped cells are formed by the division of one cell.

The term “biofilm” as used herein refers to an adherent layer of microbial cells embedded in a polymer matrix secreted by the cells. Biofilms can develop on living tissues, on catheters, in water pipes, on prostheses etc. Pathogens in biofilms may be resistant to antimicrobial agents and to the immune system.

The term “carrier” or “colonized individual” as used herein refers to a person in whom organisms are present and may be multiplying, but who shows no clinical response to their presence. The carrier state may be permanent, with the organism always present; intermittent, with the organism present for various periods; or temporary, with carriage for only a brief period. [Medical Microbiology, 4th Ed., Samuel Barron, Ed. University of Texas Medical Branch, Galveston, TX (1996): Chapter 5, p. 148].

The term “cell cycle” as used herein refers to the events which occur during growth in a single cell., including (i) the initiation of DA replication from at least one chromosomal origin; (ii) the replication of chromosome(s); (iii) the segregation of chromosomes; (iv) the formation of a septum or intercell partition; and (v) cell division. [Singleton, P., Sainsbury, D. Dictionary of Microbiology and Molecular Biology, 3rd Ed. Revised, John Wiley & Sons, Ltd., England (2006)].

The term “cell wall” as used herein refers to the structure in most algae, archaeans, bacteria and fungi that forms a (usually rigid) layer eternal to the cytoplasmic membrane and which is responsible for the shape of the organism. It protects the protoplast from mechanical damage, osmotic lysis etc. and may serve as a permeability barrier to antibiotics and other substances. Microbial cell walls differ greatly in structure and composition, according to type and species.

The term “chain of infection” as used herein refers to the three major links in disease occurrence: the etiologic agent; the method of transmission, and the host. Environmental factors can affect any link in the chain of infection. For example, temperature can assist or inhibit multiplication of organisms at their reservoir; air velocity can assist the airborne movement of droplet nuclei; low humidity can damage mucus membranes; and ultraviolet radiation can kill the microorganisms. [Medical Microbiology, 4th Ed., Samuel Barron, Ed. University of Texas Medical Branch, Galveston, TX (1996): Chapter 5, p. 145-9].

The term “conjugation” as used herein refers to a process in which genetic material is transferred from one microorganism to another involving a physical connection between the two cells.

The term “conjugative transposition” as used herein refers to conjugation mediated by a conjugative transposon, meaning a transposable element that encodes not only the functions necessary for transposition but also resistance to antibiotics, heavy metals or both, toxin production, etc. Independently of a conjugative plasmid, the transposon can be transferred from a donor bacterial cell to a recipient bacterial cell. [Singleton, P., Sainsbury, D. Dictionary of Microbiology and Molecular Biology, 3rd Ed. Revised, John Wiley & Sons, Ltd., England (2006)].

The term “differential diagnosis” refers to efforts to establish the cause of an infection, including a careful history, physical examination and appropriate laboratory studies, including selection of appropriate specimens for microbiologic examination.

The term “disease” as used herein refers to overt clinical manifestation of an infection. In an inapparent (subclinical) infection, an immune response can occur without overt clinical disease. [Medical Microbiology, 4th Ed., Samuel Barron, Ed. University of Texas Medical Branch, Galveston, TX (1996): Chapter 5, p. 148].

The term “dissemination” as used herein is the movement of an infectious agent from a source directly into the environment; when infection results from dissemination, the source, if an individual, is referred to as a dangerous disseminator. [Medical Microbiology, 4th Ed., Samuel Barron, Ed. University of Texas Medical Branch, Galveston, TX (1996): Chapter 5, p. 148].

The term “etiologic agent” as used herein refers to any microorganism that can cause infection. The pathogenicity of an etiologic agent is its ability to cause disease; pathogenicity is further characterized by describing the organism's virulence and invasiveness. Factors that should be considered in describing the etiologic agent include the infecting dose, method of transmission, site of entrance, host defenses, host species, specificity; antigenic composition, antibiotic sensitivity, resistance transfer plasmids and enzyme production. [Medical Microbiology, 4th Ed., Samuel Barron, Ed. University of Texas Medical Branch, Galveston, TX (1996): Chapter 5, p. 148].

The term “eukaryotic cell” as used herein refers to a cellular organism having a membrane bound nucleus within which the genome of the cell is stored as chromosomes composed of DNA; eukaryotic organisms include algae, fungi, protozoa, plants and animals.

The term “fungi” as used herein refers to a group of diverse, widespread unicellular (e.g., yeast) and multicellular eukaryotic organisms (e.g., molds), lacking chlorophyll and usually bearing spores and often filaments. Typically, fungal cells contain cell walls containing chitin at some point in their life cycle.

The term “genome” as used herein refers to the complete set of genetic information contained in a haploid set of chromosomes.

The term “genotype” as used herein refers to the genetic information contained in the entire complement of alleles.

The term “genus” as used herein refers to a taxonomic group directly above the species level, forming the principal subdivisions of the family.

The term “Gram stain” as uses herein refers to a differential staining procedure in which bacteria are classified as Gram-negative or Gram-positive, depending on whether they retain or lose the primary stain when subject to treatment with a decolorizing agent; the staining procedure reflects the underlying structural differences in the cell walls of Gram-negative and Gram-positive bacteria.

The term “growth” as used herein refers, In a single living cell, to a coordinated increase in the mass of essential cell components leading, typically to progress through the cell cycle; in a population of cells a coordinated increase in biomass, or an increase in the number of cells in the population.

The term “growth curve” as used herein refers to a plot of the increase in the size or number of microorganisms against the elapsed time.

The term “growth rate” as used herein refers in the number of microorganisms per unit time.

The term “host” as used herein refers to the third link in the chain of infection. The organism may enter the host through the skin, mucus membranes, lungs, gastrointestinal tract, or genitourinary tract, and it may enter fetuses through the placenta. The resulting disease often reflects the point of entrance, but not always. Development of disease in a host reflects agent characteristics and is influenced by host defense mechanisms. Nonspecific defense mechanism include the skin, mucus membranes, secretions, excretions, enzymes, the inflammatory response, genetic factors, hormones, nutrition, behavioral patterns, and the presence of other diseases. Specific defense mechanisms or immunity may be natural, resulting from exposure to the infectious agent, or artificial, resulting from active or passive immunization. [Medical Microbiology, 4th Ed., Samuel Barron, Ed. University of Texas Medical Branch, Galveston, TX (1996): Chapter 5, p. 148-9].

The term “infecting dose” as used herein refers to the number of organisms necessary to cause disease. [Medical Microbiology, 4th Ed., Samuel Barron, Ed. University of Texas Medical Branch, Galveston, TX (1996): Chapter 5, p. 148].

The term “infection” as used herein refers to the replication of organisms in the tissue of a host. Infections may be caused by bacteria, viruses, fungi and parasites. The chain of infection includes the three factors that lead to infection: the etiologic agent; the method of transmission, and the host. Manifestations of infection depend on many factors, including the site of acquisition or entry of the microorganism, organ or system tropisms of the microorganism; microbial virulence; the age, sex and immunologic status of the patient, underlying disease or conditions, and the presence of implanted prosthetic devices or materials. The signs and symptoms of infection may be localized, or they may be systemic. [Medical Microbiology, 4th Ed., Samuel Barron, Ed. University of Texas Medical Branch, Galveston, TX (1996): Chapter 5, p. 148, Chapter 10, pp. 153-154].

The term “infection control” as used herein refers to measures that prevent and contain the spread of infectious diseases.

The term “infectiousness” as used herein refers to the transmission of organisms from a source or reservoir to a susceptible individual. A human may be infective during the preclinical, clinical, postclinical or recovery phase of an illness. The term “incubation period” as used herein refers to the interval in the preclinical period between the time at which the causative agent first infects the host and the onset of clinical symptoms; during this time, the agent is replicating. The individual may be infective during the convalescent phase, as in diphtheria, or may become an asymptomatic carrier and remain infective for a prolonged period. [Medical Microbiology, 4th Ed., Samuel Barron, Ed. University of Texas Medical Branch, Galveston, TX (1996): Chapter 5, p. 146-47].

The term “invasiveness” with respect to an organism as used herein refers to its ability to invade hum an cells and tissues and to multiply on or within them to establish an infection within the body.

The term “nosocomial disease” as used herein refers to a disease acquired in hospital.

The term “microbes” as used herein refers to microscopic organisms or microorganisms. The term “microorganism: as used herein refers to microscopic organisms within the categories Algae, Archaea, Bacteria, Fungi (including Lichens), protozoa, viruses and subviral agents.

The term “Next Generation Sequencing” or “NGS” as used herein refers to a method of parallel sequencing. For instance, a nucleic acid (e.g., DNA) sample is obtained and prepared into a library (meaning a collection of nucleic acid fragments from the sample). The library is prepared by fragmenting the DNA or RNA sample. Fragmentation can be performed by physical (e.g., sheared by acoustics, nebulization, centrifugal force, needles, or hydrodynamics) or enzymatic (e.g., site-specific or non-specific nucleases) methods. In some embodiments, the fragments are about 200 bp, about 20 bp, about 300 bp, or about 350 bp in length. The DNA or RNA samples are repaired at the ends (e.g., blunt-ended) and then A-tailed (e.g., an adenosine is added to the 3′ end resulting in an overhang). Adapters are ligated to each end. Adapters include sequences, such as barcodes, restriction sites, and primer sequences.

The term “pathogen” as used herein refers to a microorganism capable of causing disease. The pathogen may be exogenous (meaning acquired from environmental or animal sources or from other persons) or endogenous (from the normal flora).

The term “plasmid” as used herein refers to extrachromosomal genetic structures that can replicate independently within a bacterial cell. The term “R plasmid” as used herein refers to a plasmid encoding for antibiotic resistance.

The term “prokaryotic” as used herein refers to cells whose genomes are not contained within a nucleus, e.g., bacterial and archaeal cells.

The term “reservoir” as used herein with respect to an organism is the site where the organism resides, metabolizes and multiples. [Medical Microbiology, 4th Ed., Samuel Barron, Ed. University of Texas Medical Branch, Galveston, TX (1996): Chapter 5, p. 148].

The term “source” of an organism is the site from which it is transmitted to a susceptible host, either directly or indirectly through an intermediary object. The reservoir and source can be the same, or can be different. [Medical Microbiology, 4th Ed., Samuel Barron, Ed. University of Texas Medical Branch, Galveston, TX (1996): 148].

The term “species” as used herein refers to a taxonomic category ranking just below a genus, which includes individuals that display a high degree of mutual similarity; the similarity can be represented as (%) between chromosomal DNA from different microbial strains.

The term “spreading of microbes” as used herein refers to transmission.

The term “standardized infection ratio” or “SIR” as used herein refers to a statistic used to track healthcare associated infections (“HAIs) over time. It compares the actual number of HAIs at each hospital to the predicted number of infections.

The term “strain” as used herein refers to a population of cells derived by asexual reproduction from a single parental cell.

The term “susceptible subject” or “susceptible host” are used interchangeably herein to refer to an individual vulnerable to developing infection when their body is invaded by an etiologic agent.

The term “taxon” as used herein refers, in a given system of biological classification, any category, consisting of one or more kinds of organism, regarded as having an identify distinct from that of any other category in that system; it may be a group of strains, species, genera, etc.

The term “taxonomy” as used herein refers to the science of biological classification that ideally reflects the evolutionary relationships between organisms. The modern approach to prokaryotic taxonomy began with molecular studies in which organisms are compared and classified according to the sequences of nucleotides in their nucleic acids. [Singleton, P., Sainsbury, D. Dictionary of Microbiology and Molecular Biology, 3rd Ed. Revised, John Wiley & Sons, Ltd., England (2006)].

The term “transduction” as used herein refers to the transfer of bacterial genes from one bacterium to another by a viral carrier (e.g., bacteriophage).

The term “transformation” as used herein refers to a mode of genetic transfer in which a naked DNA fragment derived from one microbial cell (typically bacterial) is taken up by another and subsequently undergoes recombination with the recipient.

The term “transmissible disease” as used herein refers to any disease that can be transmitted from one individual to another by any means; transmissible disease is more inclusive, broader, than infectious disease. The term “viable” as used herein refers to the ability to grow and reproduce.

The term “transmission” as used herein refers to the method by which a pathogenic agent goes from a source to a host. Transmission involves the following stages: escape from the host or reservoir of infection (meaning where the infectious agent normally lives and multiples); transport to the new host; and entry to the new host. The four major methods of transmission are by contact, by common vehicle, by air or via a vector. [Medical Microbiology, 4th Ed., Samuel Barron, Ed. University of Texas Medical Branch, Galveston, TX (1996): Chapter 5, p. 148].

In “transmission by contact”, the agent is spread directly, indirectly, or by airborne droplets. Direct contact transmission (also referred to as person-to-person contact) takes place when organisms are transmitted directly from the source to the susceptible host without involving an intermediate object. Indirect transmission occurs when the organisms are transmitted from a source, either animate or inanimate, to a host by means of an inanimate object. Droplet spread refers to organisms that travel through the air very short distances, i.e., less than 3 feet from a source to a host. Therefore, the organisms are not airborne in the true sense.

“Common-vehicle transmission” refers to agents transmitted by a common inanimate vehicle, with multiple cases resulting from such exposure.

The term “airborne transmission” as used herein refers to infection spread by droplet nuclei or dust. Droplet nuclei are the residue from the evaporation of fluid from droplets, are light enough to be transmitted more than 3 feet from the source, and may remain airborne for prolonged periods.

The term “vector borne transmission” may be external or internal. External, or mechanical, transmission occurs when organisms are carried mechanically on the vector (arthropods). Internal transmission occurs when the organisms are carried within the vector. If the pathogen is not changed by its carriage within the vector, the carriage is called harborage. The other form of internal transmission is termed biologic vector borne transmission. In this form, the organism is changed biologically during its passage through the vector. [Medical Microbiology, 4th Ed., Samuel Barron, Ed. University of Texas Medical Branch, Galveston, TX (1996): Chapter 5, p. 148].

The terms “variants”, “mutants”, and “derivatives” are used herein to refer to nucleotide sequences with substantial identity to a reference nucleotide sequence. The differences in the sequences may by the result of changes in sequence or structure. Natural changes may arise during the course of normal replication or duplication in nature of the particular nucleic acid sequence.

The term “virulence” and its other grammatical forms as used herein refers to the severity of infection in the host, which can be expressed by describing the morbidity (meaning incidence of disease) and mortality (death rate) of the infection. No microorganism is assuredly avirulent. An organism may have very low virulence, but if the host is highly susceptible, as when therapeutically immunosuppressed, infection with that organism may cause disease. [Medical Microbiology, 4th Ed., Samuel Barron, Ed. University of Texas Medical Branch, Galveston, TX (1996): Chapter 5, p. 147].

The term “virulence factors” as used herein refers to inherent properties of disease-causing microorganisms that enhance their pathogenicity, allowing them to invade human tissue and disrupt normal body functions.

The term “virulent pathogen” as used herein refers to an organism with specialized properties that enhance its ability to cause disease.

The term “virus” as used herein refers to a noncellular entity that consists minimally of protein and nucleic acid and that can replicate only after entry into specific types of living cells; it has no intrinsic metabolism and its replication depends on the direction of a cellular metabolism by the viral genome. Within the host cell, viral components are synthesized separately and are assembled intracellularly to form mature, infectious viruses.

The term “whole genome sequencing” or “WGS” as used herein refers to a technique for directly sequencing all available DNA materials from a sample. Unlike focused approaches, such as targeted sequencing, WGS delivers a comprehensive view of the entire genome. It captures both large and small variants that might be missed with targeted approaches, provides a high-resolution, base-by-base view of the genome, provides high taxonomic resolution and shows a complete microbial profile of the microbial community besides generating information on the functional genes present in the microbes.

### Methods and Systems for Dynamic Infection Tracking

Some embodiments provide a system and method that captures and analyzes electronic medical information with little time delay (e.g., every 1-2 days, daily, twice a day, hourly, in nearly real-time, in real-time etc.) to visualize and dynamically map infections in healthcare environments and provide infection control intelligence to prevent, locate and enable proactive management of outbreaks and improve patient safety. Some embodiments of methods and systems provide infectious disease professionals with tools to identify, track, and manage infectious disease outbreaks in health care environments. Such systems and methods may be referred to herein as dynamic infection monitoring and tracking systems and methods or dynamic infection propagation detection system and methods herein. In some embodiments, systems and methods are configured to generate interactive electronic maps based on information regarding health care facilities pulled from one or more data sources that include patient location and movement and patient interactions with healthcare providers and medical instruments. In some embodiments, systems and methods will also provide and track bacterial data regarding infections. In some embodiments, the bacterial data include one or both of antibiograms and genetic characterization. In some embodiments, the systems and methods are capable of monitoring across a broad geographical space. In some embodiments, the systems and methods monitor multiple different healthcare facilities that may be separated geographically. In some embodiments, systems and methods employ interactive analysis of multiple variables to provide infection control practitioners with updated periodic (e.g., daily) or on demand results with customized visualization tools for different portions (e.g., floors) of one or more healthcare system facilities to identify and locate all encounters or each encounter, over time and space, and evaluate and prioritize suspected cases of transmission. In some embodiments, systems and methods provide guidance regarding where to implement infection control interventions. In some embodiments, in parallel, each population of infecting bacteria may be captured for whole genome sequencing to confirm or refute microbial strain identity among cases of suspected transmission and create a genomic library that can be used to identify rapid genotyping tools to control the most prevalent healthcare system pathogens. In some embodiments, the period (e.g., daily, twice a day, hourly, etc.) electronic reporting, enables a database of the movement and healthcare interactions associated with all infected patients with each type of infection (e.g., urine, wound, respiratory or blood) to be built and linked to the genetically sequenced pathogen. This database of movement and healthcare interacts may enable the use of artificial intelligence to dissect and identify hot-spots of transmission and trends that increase the risk of transmitting an infection with the goal of improving care for infected patients and reducing the chance of spreading hospital pathogens.

In healthcare facilities, preventing the spread of infections is of paramount importance to ensure the safety and well-being of both patients and healthcare workers. Timely detection of infection propagation is critical for implementing effective containment measures. Some embodiments of systems and methods provide a reduced time lag, responsive infection propagation detection system using a dataset from an electronic health record management system containing information about patients, healthcare workers, and instruments, along with their interactions and infection records.

### Dynamic Infection Control

Genomic clusters defined by whole genome sequencing are the established standard to confirm cases of transmission. This includes sequencing all pathogens and investigating the patients who have the identical infecting microbial strain. Currently, the logistics, speed and cost of whole genome sequencing of all infecting pathogens prevent its use for dynamic infection control on a sufficiently short time scale to effectively and proactively address the spread of infection. Obtaining infection information with little time lag and dynamic infection analysis including patient mapping, movement, and identification of healthcare provider contacts and instrument or device contacts can provide early identification suspected cases of transmission and guide and prioritize the microbial infection strains that need genomic evaluation.

### Periodic and/or On Demand Patient Tracking

Some embodiments including periodic (e.g., daily, hourly, etc.) and/or on demand patient tracking based on information obtained from or generated by an electronic health record management system. In some embodiments, the tracking is every minute, every hour, every 2 hours, every 3 hours, every 4 hours, every 5 hours, every 6 hours every 7 hours, every 8 hours, every 9 hours, every 10 hours, every 11 hours, every 12 hours, every 13 hours, every 14 hours, every 15 hours, every 16 hours, every 17 hours, every 18 hours, every 20 hours, every 21 hours, every 22 hours, every 23 hours, or every 24 hours. In some embodiments, the tracking is at least every minute, every hour, every 2 hours, every 3 hours, every 4 hours, every 5 hours, every 6 hours every 7 hours, every 8 hours, every 9 hours, every 10 hours, every 11 hours, every 12 hours, every 13 hours, every 14 hours, every 15 hours, every 16 hours, every 17 hours, every 18 hours, every 20 hours, every 21 hours, every 22 hours, every 23 hours, or every 24 hours. In some embodiments, the tracking is about once a day, about twice a day, about 3 times a day, about 4 times a day, about 5 times a day, about 6 times a day, about 7 times a day, about 8 times a day, about 9 times a day, about 10 times a day, about 11 times a day, about 12 times a day, about 13 times a day, about 14 times a day, about 15 times a day, about 16 times a day, about 17 times a day, about 18 times a day, about 19 times a day, about 20 times a day, about 21 times a day, about 22 times a day, about 23 times a day, or about 24 times a day. In some embodiments, the tracking is at least about once a day, about twice a day, about 3 times a day, about 4 times a day, about 5 times a day, about 6 times a day, about 7 times a day, about 8 times a day, about 9 times a day, about 10 times a day, about 11 times a day, about 12 times a day, about 13 times a day, about 14 times a day, about 15 times a day, about 16 times a day, about 17 times a day, about 18 times a day, about 19 times a day, about 20 times a day, about 21 times a day, about 22 times a day, about 23 times a day, or about 24 times a day.

The patient tracking may include information regarding each patient having an infection. For each patient having an infection, the daily patient tracking may include some or all of: a date/time of infection, a length of infection, a time of infection, and pathogen and antibiotic susceptibility, a geographic and/or facility location, any healthcare worker contacts with location and temporal information, and any durable medical device or instrument contacts with location and temporal information. The patient tracking enables infection control practitioners to understand every interaction event including interactions with healthcare workers capturing what the interaction was, what time it occurred, where the interaction occurred and what healthcare workers and/or instruments were involved.

In some embodiments, the tracking is every minute, every hour, every 2 hours, every 3 hours, every 4 hours, every 5 hours, every 6 hours every 7 hours, every 8 hours, every 9 hours, every 10 hours, every 11 hours, every 12 hours, every 13 hours, every 14 hours, every 15 hours, every 16 hours, every 17 hours, every 18 hours, every 20 hours, every 21 hours, every 22 hours, every 23 hours, or every 24 hours. In some embodiments, the tracking is at least every minute, every hour, every 2 hours, every 3 hours, every 4 hours, every 5 hours, every 6 hours every 7 hours, every 8 hours, every 9 hours, every 10 hours, every 11 hours, every 12 hours, every 13 hours, every 14 hours, every 15 hours, every 16 hours, every 17 hours, every 18 hours, every 20 hours, every 21 hours, every 22 hours, every 23 hours, or every 24 hours. In some embodiments, the tracking is about once a day, about twice a day, about 3 times a day, about 4 times a day, about 5 times a day, about 6 times a day, about 7 times a day, about 8 times a day, about 9 times a day, about 10 times a day, about 11 times a day, about 12 times a day, about 13 times a day, about 14 times a day, about 15 times a day, about 16 times a day, about 17 times a day, about 18 times a day, about 19 times a day, about 20 times a day, about 21 times a day, about 22 times a day, about 23 times a day, or about 24 times a day. In some embodiments, the tracking is at least about once a day, about twice a day, about 3 times a day, about 4 times a day, about 5 times a day, about 6 times a day, about 7 times a day, about 8 times a day, about 9 times a day, about 10 times a day, about 11 times a day, about 12 times a day, about 13 times a day, about 14 times a day, about 15 times a day, about 16 times a day, about 17 times a day, about 18 times a day, about 19 times a day, about 20 times a day, about 21 times a day, about 22 times a day, about 23 times a day, or about 24 times a day.

### Data Collection

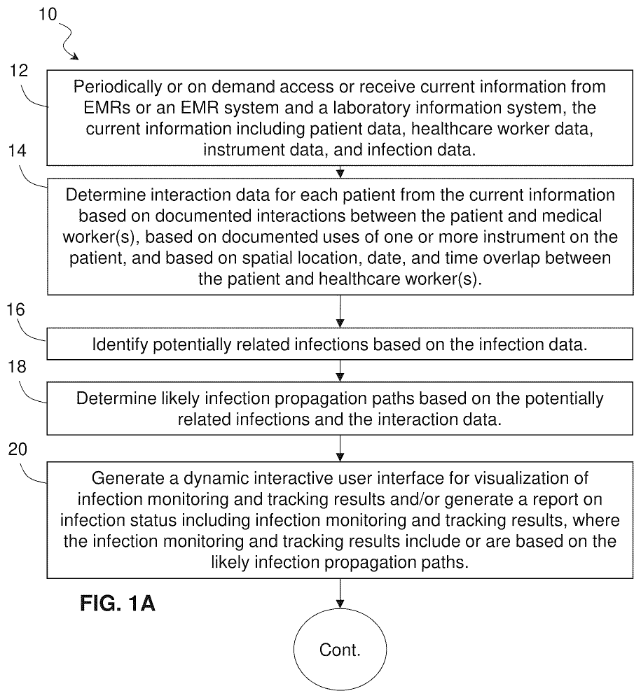

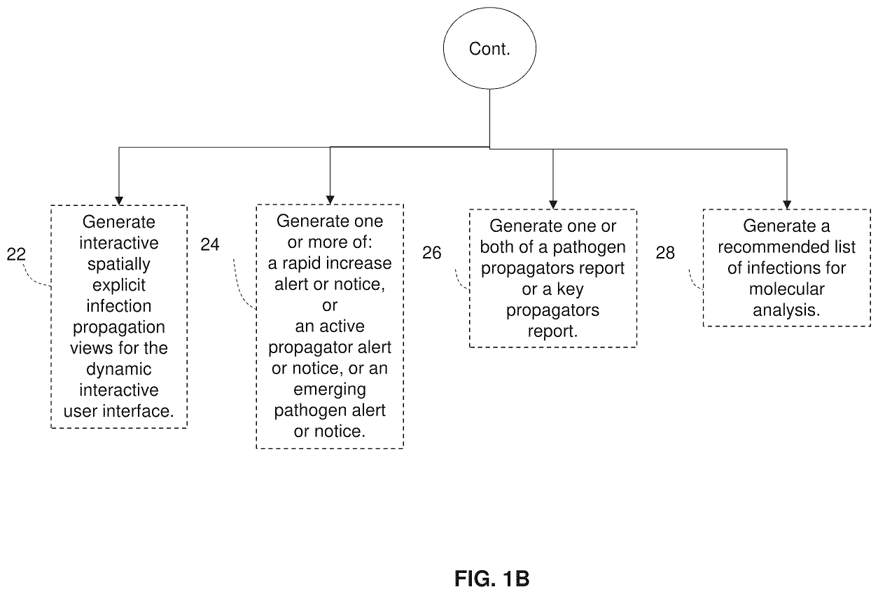

FIG. 1A is a flow chart of a method 10 for infection tracking in accordance with some embodiments. Current information is accessed or received, at least in part, from electronic medical records (EMR) or from an electronic medical record (EMR) management system (e.g., EPIC by Epic Systems Corp., Athenahealth EMR system by Athenahealth, Inc., DrChrono EMR system by EverHealth Solutions Inc., AdvancedMD EHR system by Advanced MD) employed by a healthcare facility or a healthcare system (step 10). In some embodiments, at least some of the current information is accessed or retrieved from a laboratory information system. In some embodiments, the laboratory information system is included in, is an add-on to, or is connected to the electronic medical record management system (e.g., Beaker Laboratory Information System by EPIC Systems Corp.). In some embodiments, the laboratory information system is independent from, but in communication with the electronic medical record management system. The accessed or received current information includes information regarding patients (e.g., a plurality of patients), healthcare workers, infection(s), and instrument(s). In some embodiments, the accessed or received current information regarding the plurality of patients includes information regarding patients that have or previously had at least one infection while in the care of the healthcare system or healthcare facility (e.g., within a relevant time period). In some embodiments, the accessed or received current information regarding the plurality of patients includes only information regarding patients that have or previously had at least one infection while in the care of the healthcare system or healthcare facility (e.g., within a relevant time period). In some embodiments, the accessed or received information regarding the plurality of patients also includes information regarding patients that do not have and have not had at least one infection while in the care of the healthcare system or healthcare facility (e.g., within a relevant time period).

The accessed or retrieved current information regarding patients (e.g., a plurality of patients), healthcare workers, infection(s), and instrument(s), which may be referred to herein as collected information, is integrated and stored in a database identified herein as an infection monitoring and tracking (IMaT) database organized as patient data, healthcare worker data, instrument data, and infection data.

### Data Integration and Preprocessing

In some embodiments, the accessed or received current information, which may be referred to herein as the collected information or the collected data, may be integrated and may be preprocessed before or after being stored in the separate IMaT database. For example, in some embodiments the accessed or received current information may be merged and cleaned to create a comprehensive data set. In some embodiments, the collected data may be normalized and standardized for consistency and accuracy. In some embodiments, missing data may be handled through imputation techniques.

In some embodiments, an instrument type may be obtained directly from the EMR system. In some embodiments, the instrument type may be deduced or inferred based on a location of the instrument, changes in location of the instrument, or other data. In some embodiments, a type of healthcare worker may be obtained directly from the EMR system. In some embodiments, the type of the heathcare worker may be deduced or inferred from the number of patients visited, distance traveled, etc.

### Data Enrichment

In some embodiments, the collected data, which may be normalized and standardized, may also be enriched to include additional features and types of information. The additional features may include, but are not limited to, any of healthcare worker contact history, heathcare worker type, instrument type, instrument contact history, contact frequency, contact duration, and geographic or spatial proximity to potential infection sources, which are also stored in the IMaT database. Contacts between patients and healthcare workers and between patients and instruments is characterized herein as interaction data. In some embodiments, interaction data also includes contacts between different patients. In some embodiments, interaction data also includes at least some contacts between different healthcare workers (e.g., when contacts between different healthcare workers and a patient overlap in time. As one example of enrichment of the data, in some embodiments the stored data is analyzed to determine at least some contacts between patients and healthcare workers and/or to determine at least some contacts between patients and instruments, producing enriched data that is interaction data. In some embodiments, healthcare worker contact history is determined by extracting and joining all spatial and temporal overlaps with patients. In some embodiments, the stored data is analyzed to determine to determine a frequency of contacts. In some embodiments, a contact frequency or interaction frequency is calculated as a total number of contact or interactions over a period of time for a patient, where each contact is between the patient and a healthcare worker or between the patient and an instrument. For an interaction that includes a healthcare worker and an instrument at the same time (e.g., the healthcare worker is bringing or using the instrument), this may be considered two contacts for the purposes of contact frequency. In some embodiments, the stored data is analyzed to determine to determine a contact duration (e.g., for a patient-healthcare worker contact and/or for a patient-instrument contact). In some embodiments, a contact frequency may be determined as a total number of contacts or interactions that two patients have in common over a period of time (e.g., each common contact or interaction would mean that both patients had an interaction with the same instrument or the same healthcare worker in the period of time). Contacts may also or alternatively be referred to as interactions herein. Thus, contact frequency may alternatively be referred to herein as interaction frequency and contact duration may alternatively be referred to as interaction duration herein.

The system retrieves, has access to or stores geographic or spatial information regarding one or more healthcare facilities or healthcare systems. In some embodiments, intra hospital spatial position/distance is derived from charts, which indicate institution, floor, and room, to get a location that is identified on a floorplan or a floor map. In some embodiments, the geographic or spatial data may be obtained in a Computer Aided Drawing (CAD) format (e.g., as a dwg file) with positions matching the names for each room listed in patient data. In some embodiments, the geographical or spatial data may have been obtained from images and converted into a suitable format for determining relative spatial positions of patient rooms and, in some embodiments, individual patient beds.

As another example for enrichment of the data, in some embodiments, geographic or spatial information regarding one or more healthcare facilities or systems is combined with or applied to patient data and one or more of healthcare worker data or instrument data. In some embodiments, geographic or spatial information regarding healthcare facilities or systems is combined with or applied to patient data and one or more of healthcare worker data or instrument data to determine a distance from a patient to a potential infection source or a separation between potential infection sources. In some embodiments, geographic or spatial information regarding healthcare facilities or systems is combined with patient data and one or more of healthcare worker data or instrument data for mapping patients and contacts onto representations of healthcare facilities or systems. The enriched data may also be stored in the IMaT database under one or more of patient data, healthcare worker data, instrument data, infection data, and contact data.

The collected data in an integrated and preprocessed form and the enriched data together can collectively be referred to as the IMaT data, which includes patient data, healthcare worker data, instrument data, infection data, and contact data/interaction data. The patient data includes, for at least each patient identified as having an infection: an identifier for the patient, demographic information, medical history, and admission information. The healthcare worker data includes, for each healthcare worker: an identifier for the healthcare worker, a role or position of the healthcare worker. In some embodiments, the heathcare worker data includes a contact history for the healthcare worker with patients and/or with instruments at the healthcare facility or in the healthcare system. In some embodiments, the contact history for the healthcare worker may be enriched information generated from the collected data. In some embodiments, the healthcare worker data includes dates and time periods that the healthcare worker was working or present at the healthcare facility or healthcare system.

In some embodiments, a tracking mechanism may be employed that enables the system to determine or sense locations of heathcare workers within the healthcare facility at different times. For example, such a tracking mechanism may be employed via an app on a mobile device. In some embodiments, the tracking mechanism may provide realtime positional monitoring of healthcare workers (e.g., via an app on a mobile device). As another example, such a tracking mechanism could include a passive RFID worn on a healthcare worker's identification tag that detects when the healthcare worker enters or exits rooms or various areas of the healthcare facility. Such information regarding healthcare worker locations at various times could be used to more accurately determine contacts with patients. Such information regarding healthcare workers at various times could also be used to more accurately determine a duration of patient contacts.

In some embodiments, a tracking mechanism may be employed that enables the system to determine or sense locations of patients within the healthcare facility at different times. Such a tracking mechanism could be in the form of an RFID tag in a patient's identification bracelet or another suitable tracking mechanism.

In some embodiments, a tracking mechanism may be employed that enables the system to determine or sense locations of instruments within the healthcare facility at different times. Such a tracking mechanism could be in the form of an RFID tag or transponder attached to or associated with the instrument.

The instrument data includes for each instrument: an identifier for the instrument and record information about the instrument including its usage including dates and time periods. In some embodiments, the instrument data may also include a cleaning schedule including dates and time where available. In some embodiments, the instrument data may include a maintenance history including date and time where available. In some embodiments, information regarding a cleaning schedule of an instrument and or maintenance history of an instrument may be obtained from a source other than an EMR system or a laboratory information system.

The interaction data includes interactions between patients, healthcare workers, and instruments including interaction dates and times, locations, and duration of interactions. In some embodiments, at least some or all of the interaction data is enriched data that is generated based on analysis of the collected data.

The infection data includes data regarding cases of infections among patients and healthcare workers including: type of infection, date of diagnosis, antibiograms where available, and genome sequencing where available. In some embodiments, the antibiogram data is obtained from a laboratory information system. In some embodiments, genome sequencing information is obtained from a laboratory information system. In some embodiments, the collected infection data identifies a microbial strain for at least some of the infections. In some embodiments, the infection data includes possible sources of infection. In some embodiments, possible sources of infection are identified. Possible sources of infection requires specific information regarding each known microbial species (e.g., methods of transmission etc.), which could be obtained from known sources (e.g., literature or known databases). After matching propagation modes with new microbial strains, it would be possible to match a propagation pattern of a new microbial strain to a propagation model of a known microbial strain or to create a new category.

Identifiers used for the patients and healthcare workers should be selected and stored in a manner that ensures that patient and healthcare worker privacy is respected and that data is anonymized and secured.

Updated current information including updated patient data, updated healthcare worker data, updated instrument data, updated interaction data, and updated infection data is accessed or generated periodically or on demand. In some embodiments, updated current information is accessed, retrieved, or generated at least every day. In some embodiments, updated current information is updated current information is accessed, retrieved, or generated every minute, every hour, every 2 hours, every 3 hours, every 4 hours, every 5 hours, every 6 hours every 7 hours, every 8 hours, every 9 hours, every 10 hours, every 11 hours, every 12 hours, every 13 hours, every 14 hours, every 15 hours, every 16 hours, every 17 hours, every 18 hours, every 20 hours, every 21 hours, every 22 hours, every 23 hours, or every 24 hours. In some embodiments, updated current information is updated current information is accessed, retrieved, or generated at least every minute, every hour, every 2 hours, every 3 hours, every 4 hours, every 5 hours, every 6 hours every 7 hours, every 8 hours, every 9 hours, every 10 hours, every 11 hours, every 12 hours, every 13 hours, every 14 hours, every 15 hours, every 16 hours, every 17 hours, every 18 hours, every 20 hours, every 21 hours, every 22 hours, every 23 hours, or every 24 hours. In some embodiments, updated current information is updated current information is accessed, retrieved, or generated about once a day, about twice a day, about 3 times a day, about 4 times a day, about 5 times a day, about 6 times a day, about 7 times a day, about 8 times a day, about 9 times a day, about 10 times a day, about 11 times a day, about 12 times a day, about 13 times a day, about 14 times a day, about 15 times a day, about 16 times a day, about 17 times a day, about 18 times a day, about 19 times a day, about 20 times a day, about 21 times a day, about 22 times a day, about 23 times a day, or about 24 times a day. In some embodiments, updated current information is updated current information is accessed, retrieved, or generated at least every minute at least about once a day, about twice a day, about 3 times a day, about 4 times a day, about 5 times a day, about 6 times a day, about 7 times a day, about 8 times a day, about 9 times a day, about 10 times a day, about 11 times a day, about 12 times a day, about 13 times a day, about 14 times a day, about 15 times a day, about 16 times a day, about 17 times a day, about 18 times a day, about 19 times a day, about 20 times a day, about 21 times a day, about 22 times a day, about 23 times a day, or about 24 times a day. In some embodiments, updated current information is received continuously or when available.

In some embodiments, interaction data is generated or updated on demand or periodically. In some embodiments, interaction data is generated or updated at least every day. In some embodiments, interaction data is generated or updated every minute, every hour, every 2 hours, every 3 hours, every 4 hours, every 5 hours, every 6 hours every 7 hours, every 8 hours, every 9 hours, every 10 hours, every 11 hours, every 12 hours, every 13 hours, every 14 hours, every 15 hours, every 16 hours, every 17 hours, every 18 hours, every 20 hours, every 21 hours, every 22 hours, every 23 hours, or every 24 hours. In some embodiments, interaction data is generated or updated at least every minute, every hour, every 2 hours, every 3 hours, every 4 hours, every 5 hours, every 6 hours every 7 hours, every 8 hours, every 9 hours, every 10 hours, every 11 hours, every 12 hours, every 13 hours, every 14 hours, every 15 hours, every 16 hours, every 17 hours, every 18 hours, every 20 hours, every 21 hours, every 22 hours, every 23 hours, or every 24 hours. In some embodiments, interaction data is generated or updated about once a day, about twice a day, about 3 times a day, about 4 times a day, about 5 times a day, about 6 times a day, about 7 times a day, about 8 times a day, about 9 times a day, about 10 times a day, about 11 times a day, about 12 times a day, about 13 times a day, about 14 times a day, about 15 times a day, about 16 times a day, about 17 times a day, about 18 times a day, about 19 times a day, about 20 times a day, about 21 times a day, about 22 times a day, about 23 times a day, or about 24 times a day. In some embodiments, interaction data is generated or updated at least every minute at least about once a day, about twice a day, about 3 times a day, about 4 times a day, about 5 times a day, about 6 times a day, about 7 times a day, about 8 times a day, about 9times a day, about 10 times a day, about 11 times a day, about 12 times a day, about 13 times a day, about 14 times a day, about 15 times a day, about 16 times a day, about 17 times a day, about 18 times a day, about 19 times a day, about 20 times a day, about 21 times a day, about 22 times a day, about 23 times a day, or about 24 times a day. In some embodiments, interaction data is generated or updated continuously or when new source information is available.

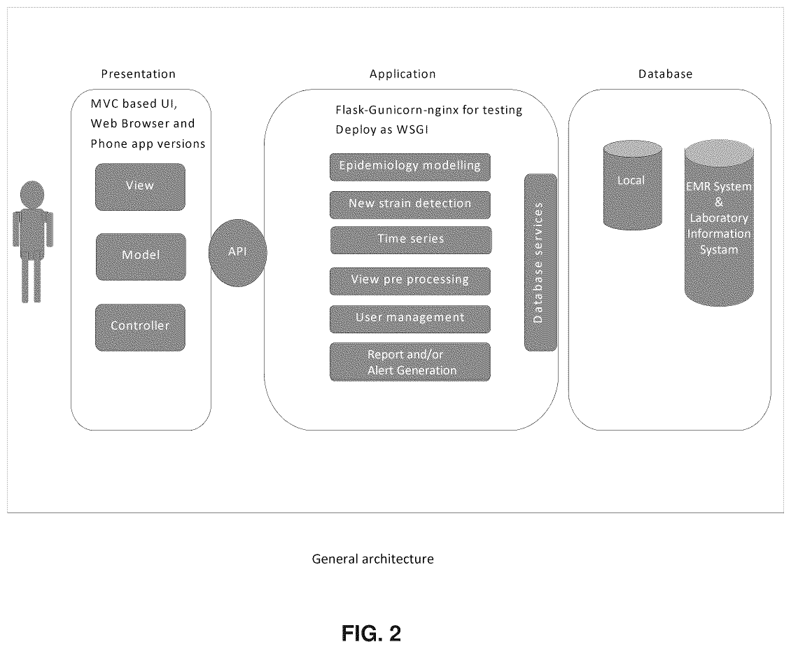

The updated current information may be processed and stored in the same database (e.g., the IMaT database) (see FIG. 2) and be enriched to form additional IMaT data. In some embodiments, information from an EMR system and/or a laboratory information system is posted or stored continuously and/or periodically (e.g., into a database onto a spreadsheet), and updated, new or changed information is accessed (e.g., retrieved from storage or pulled from the spreadsheet) to update the separate database (e.g., the IMaT database) periodically or on demand (see FIG. 2).

In some embodiments, the IMaT database is updated at least every day. In some embodiments, the IMaT database is updated every minute, every hour, every 2 hours, every 3 hours, every 4 hours, every 5 hours, every 6 hours every 7 hours, every 8 hours, every 9 hours, every 10 hours, every 11 hours, every 12 hours, every 13 hours, every 14 hours, every 15 hours, every 16 hours, every 17 hours, every 18 hours, every 20 hours, every 21 hours, every 22 hours, every 23 hours, or every 24 hours. In some embodiments, the IMaT database is updated at least every minute, every hour, every 2 hours, every 3 hours, every 4 hours, every 5 hours, every 6 hours every 7 hours, every 8 hours, every 9 hours, every 10 hours, every 11 hours, every 12 hours, every 13 hours, every 14 hours, every 15 hours, every 16 hours, every 17 hours, every 18 hours, every 20 hours, every 21 hours, every 22 hours, every 23 hours, or every 24 hours. In some embodiments, the IMaT database is updated about once a day, about twice a day, about 3 times a day, about 4 times a day, about 5 times a day, about 6 times a day, about 7 times a day, about 8 times a day, about 9 times a day, about 10 times a day, about 11 times a day, about 12 times a day, about 13 times a day, about 14 times a day, about 15 times a day, about 16 times a day, about 17 times a day, about 18 times a day, about 19 times a day, about 20 times a day, about 21 times a day, about 22 times a day, about 23 times a day, or about 24 times a day. In some embodiments, the IMaT database is updated at least about once a day, about twice a day, about 3 times a day, about 4 times a day, about 5 times a day, about 6 times a day, about 7 times a day, about 8 times a day, about 9 times a day, about 10 times a day, about 11 times a day, about 12 times a day, about 13 times a day, about 14 times a day, about 15 times a day, about 16 times a day, about 17 times a day, about 18 times a day, about 19 times a day, about 20 times a day, about 21 times a day, about 22 times a day, about 23 times a day, or about 24 times a day. In some embodiments, the IMaT database is updated continuously or when available.

Current information accessed or retrieved and data stored in the IMaT database is not limited to the categories of information, types of information, or specific items of information described herein. One of ordinary skill in the art in view of the present disclosure will appreciate that may additional categories of information, types of information, or specific items of information may be accessed or retrieved, stored in the IMaT database and used in analysis and models. In some embodiments, a user may specify additional information to be collected. In some embodiments, data may be accessed or retrieved related to the use of one or more types instruments that have an associated increased risk of infection (e.g., Foley catheters).

FIG. 2 schematically depicts an example general architecture in accordance with some embodiments. In some embodiments, current information and data stored in the separate database (e.g., the IMaT database) is used by an application employed via database services for new microbial strain detection, time series analysis, epidemiology modeling, and report and/or alert generation (see FIG. 2). In some embodiments, the application enables pre-processing to be viewed and provides user management.

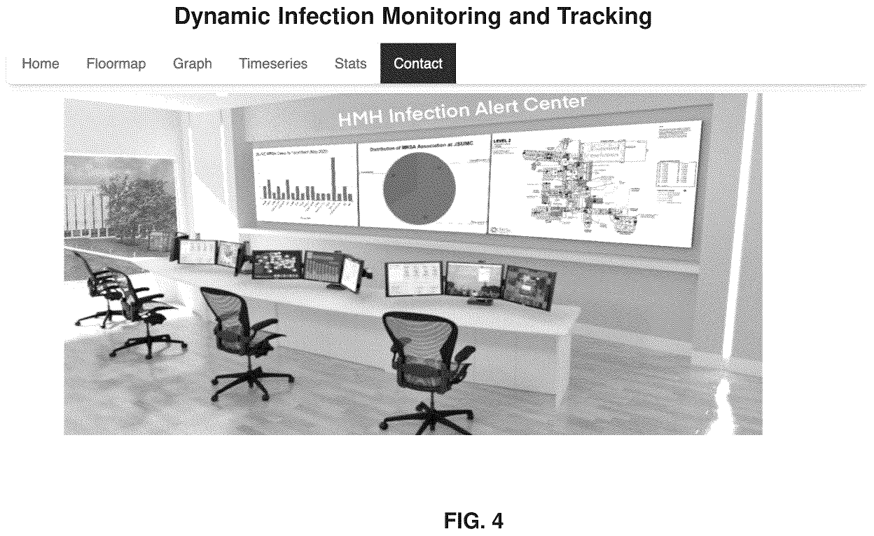

In some embodiments, the system provides a user interface (e.g., a graphical user interface) with the application (e.g., via an application programming interface (API)) that enables the user to view and interact with infection tracking results and analysis. In some embodiments, the GUI enables a user to interact with dynamic visual representations for infection tracking. In come embodiments, the presentation may be an MVC based user interface that includes internal representations of information (Model), the interface (View) that presents information to and accepts input from the user, and (Controller) software linking the two. An exemplary user interface for infection tracking results and analysis is described below with respect to FIG. 4.

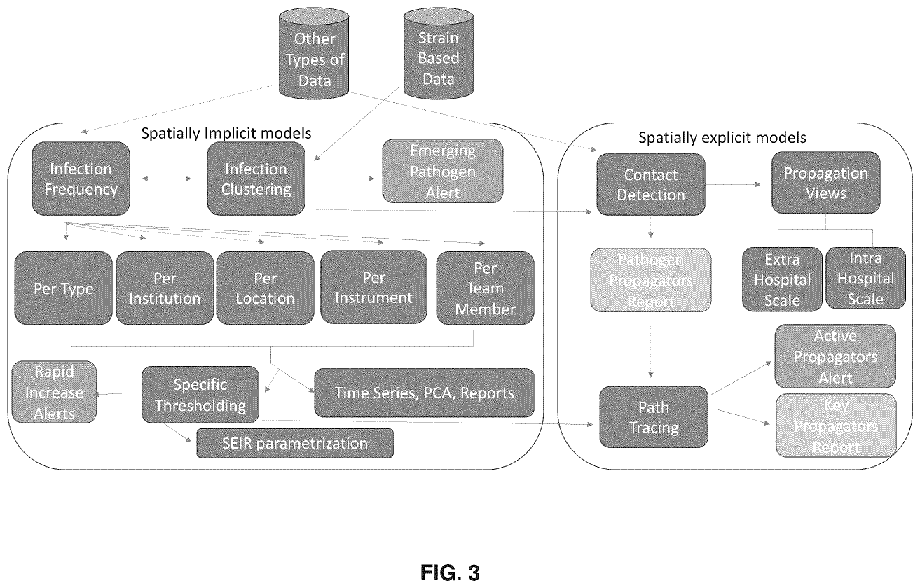

In some embodiments, the current data in the separate database (e.g., IMaT database), which may be normalized and standardized data and may include enriched data, may be grouped into microbial strain-based data and other types of data (see FIG. 3). Microbial strain-based data is data received from or obtained from a report or testing on an infection (e.g., antibiotic resistances, genotyping, growth rate, etc.). Other types of data can include, but are not limited to, patient data, healthcare worker data, instrument data, and contact data.

Some methods and systems described herein employ both spatially explicit models and spatially implicit models for analysis. Spatially explicit models are computational models that incorporate spatial information to simulate the spread of infectious diseases within a population. They take into account the physical locations of individuals within a defined area. An example of a use of spatially explicit models is mapping disease spread. The system may use spatially explicit models to generate visual representations of how an infection spreads spatially or geographically over time. A spatially explicit model may integrating data on patient density, mobility patterns, and other factors to predict how an infection might propagate through different areas. Another example of use of a spatially explicit model is for identifying high-risk areas. Identification of high-risk areas can retrospectively help design interventions such as quarantine measures, or prioritization of healthcare resources during outbreaks to mitigate the spread of infection. Another example of a use of a spatially explicit model is for modelling of transmission dynamics. Such a model enables a user to explore how environmental factors, such as distance, construction characteristics, building topology, climate or urbanization, influence the spread of an infection. Another example of use of a spatially explicit model is for generating simulated outcomes under various scenarios, assess which measures are most likely to reduce transmission rates and minimize the overall impact of an outbreak. This provides information regarding likely success various intervention strategies under consideration. This may enable decision-makers to efficiently identify areas with the greatest need, and may also inform decisions about the most efficient or cost-effective strategies for containing an outbreak.

An example of a small scale use of a spatially explicit model could include any of weighting the likelihood of contagion by the distance between patients, displaying infections on a floormap, and tracing the distance traversed by health care workers on the map. There are multiple options for predeveloped models at the large scale (e.g., Agent-Based Models (ABMs), Spatial Trans Models (STMs) and Geographic Information System (GIS) based models).

Generally speaking, spatially implicit propagation models do not consider or do not focus on the spatial distribution or location of patients. Instead, spatially implicit propagation models focus on the specific interactions between individuals (e.g. patients, or patients and healthcare workers). These models typically aggregate individuals (e.g., patients) into categories based on relevant characteristics such as age, susceptibility, or infection status. One type of spatially implicit propagation models compartmental models. Examples of compartmental models include SIR (Susceptible-Infectious-Recovered) or SEIR (Susceptible-Exposed-Infectious-Recovered) models. In these models, a population is divided into compartments representing different disease states, and individuals (e.g., patients) transition between these compartments based on specified transmission dynamics. These models track the flow of individuals through compartments over time to simulate the spread of infectious diseases within a population.

For example, in some embodiments, a susceptible-exposed-infectious-recovered (SEIR) model is employed for analysis and prediction of infection dynamics. Such a SEIR model analysis may include determining the best fitting parameters for a SEIR model per microbial species and per microbial strain from previous data. The model may be fitted at different scales, e.g., at the scale of a floor, at the scale of a zone, at the scale of a healthcare facility, at the scale of a healthcare system, etc. Alternatively or additionally, such a SEIR model analysis may include determining parameters for likelihood of spatial propagation, which are fitted using machine learning optimization. Whenever new data is incorporated into the SEIR model for a microbial species or microbial strain, it may be tested to determine whether if fits the previous SEIR model.

Another type of spatially implicit propagation models employs systems of ordinary or partial differential equations that describe the rates of change in the numbers of individuals in each compartment over time. These equations incorporate parameters such as transmission rates, recovery rates, and population demographics to capture the dynamics of infection transmission and progression. One difference between differential equation based models and SEIR models is that the differential equation based models are usually continuous probabilistic models. This may be particularly beneficial when there is a need to integrate this type of model with spatially explicit models. For example, one could employ one differential equation based model per location and also employ distance based parameter of propagation between locations.

In some embodiments, spatially implicit propagation models are generated from microbial strain-based data and other types of data. In some embodiments, spatially explicit propagation models are generated from other types of data. In some embodiments, spatial explicit models are also based on the results of spatially implicit modelling.

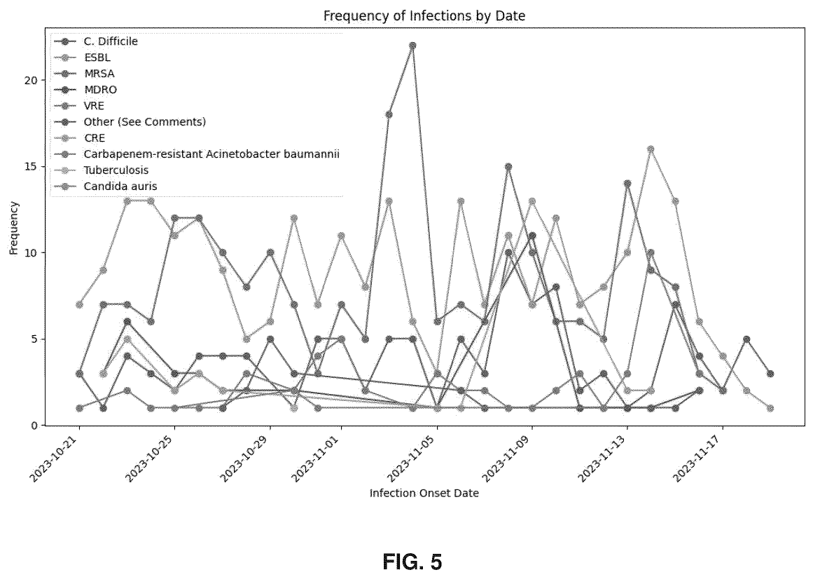

Interaction data for each patient is determined from the current information based on documented interactions between the patient and medical worker(s), based on documented uses of one or more instrument on the patient, and based on spatial location, date, and time overlap between the patient and healthcare worker(s) (FIG. 1A, step 14). For example, patient contacts can be determined for each patient based on location, date, and time overlap between the patient and healthcare workers, which is spatially explicit information, as well as based on documented interactions between the patient and healthcare workers, and on documented use of an instrument on the patient. Other types of data, such as patient data, can also be used in spatially implicit models for determining infection frequency and infection clustering in accordance with some embodiments. Infection frequency may be categorized per type, per institution, per location, per instrument and/or per healthcare worker in some embodiments. The infection frequency can also be used for generation of time series analysis and graphical representations, in principal component analysis, and in reports. FIG. 5 shows an example of patient-based infection frequency data employed in time series analysis. FIG. 5 is a graph of frequency of different types of infections in a healthcare system or facility by date. The graphs shows what appears to be a significant increase in cases of Methicillin-resistant Staphylococcus aureus (MRSA) on about Nov. 5, 2023.

The method includes identifying potentially related infections, which are referred to as “like” infections herein, based on the infection data (FIG. 1A, step 16). Infections of the same type and the same microbial strain are “like” infections or potentially related infections. Infections of the same type with microbial strains that are only slightly different may be determined to be potentially related infections. In some embodiments, microbial strain information for infections of interest has been provided in the collected information. In, some embodiments, microbial strain information has not been provided for all infections of interest or the microbial strains of different infections are only slightly different and additional analysis is needed to identify potentially related infections.

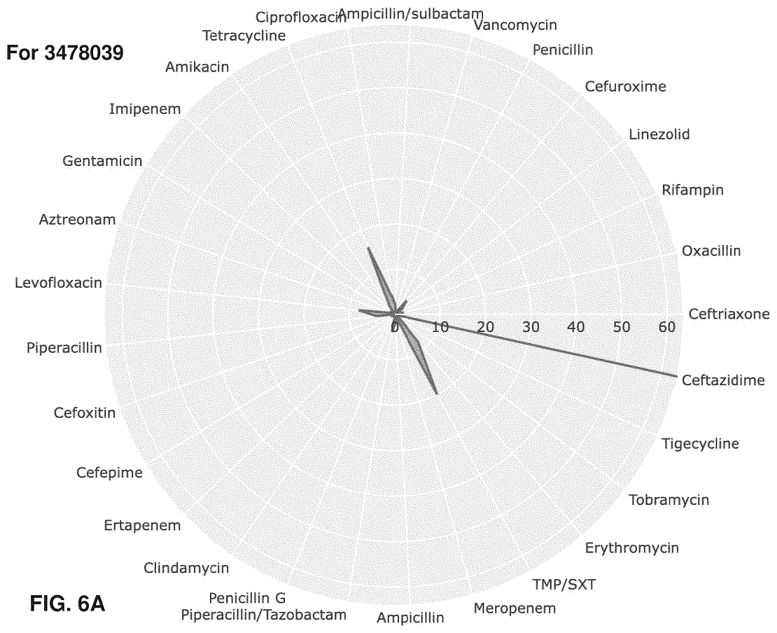

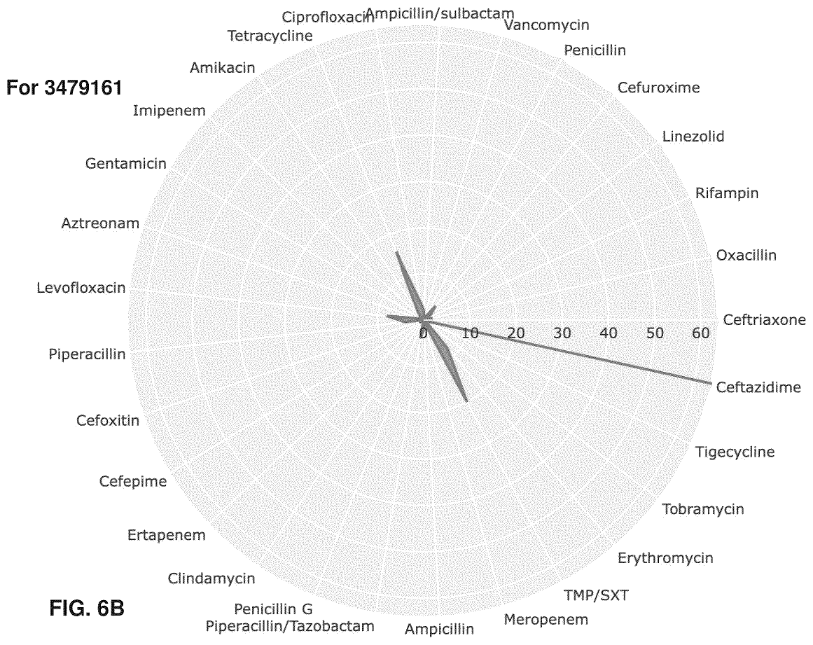

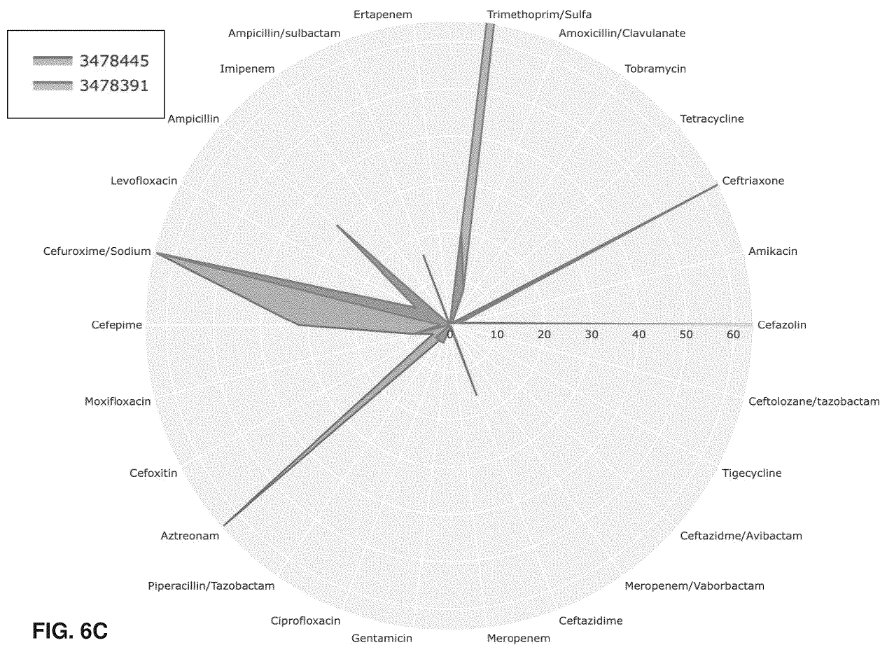

Strain-based data may be employed in spatially implicit models. In some embodiments, microbial strain information for infections of interest has been provided in the collected information. For example, comparison of antibiograms for infections from different patients may be used to aid in determining if different patients have a same microbial strain or a similar microbial strain for infection clustering purposes and for identification of potentially related infections. Examples of antibiograms for two different infection populations are graphically displayed in FIGS. 6A (population 3478039) and 6B (population 3479191) and are superimposed on the same graph in FIG. 6C. In some embodiments, clustering analysis is performed to identify potentially related infections. In some embodiments, clustering analysis is based on one of the spatially implicit models.

In some embodiments, a clustering analysis includes building a vector from data for a microbial strain. In some embodiments, dimensionality reduction techniques (e.g., principal component analysis (PCA), t-distributed stochastic neighbor embedding (tSNE), etc.) clustering methods may be employed with clustering methods. For example, the dimensionality reduction techniques may be applied to the vectors for microbial strain. In some embodiments, the dimensionality reduction techniques using microbial strain distance combined with clustering methods and using any other feature as category target may be employed. Dimensionality reduction techniques like PCA or TSNE may aid in visualizing the microbial strain. Different clustering techniques may be employed, e.g., centroid-based clustering, density-based clustering, distribution-based clustering, and/or hierarchical clustering. Thresholds for each clustering method based be set or adjusted on a previous definition of a microbial strain for a given microbial species. The clustering may identify new cluster or outliers, which could be new pathogen strains and would get priority for sequencing. The distance between two microbial strains define their likelihood to be “the same”, but thresholding specific to each microbial species is needed (e.g., antibiogram data doesn't define sameness in C. difficile, but multilocus sequence typing (MLST) can be employed to differentiate strains of C. difficile).

Both the patient-based and microbial strain-based data can be used in spatially implicit models in some embodiments. In some embodiments, information generated in the spatially implicit models may be also be employed or used as input for the spatially explicit models.

In some embodiments, likely infection propagation paths are determined based on potentially related infections and interaction data (see FIG. 1A, step 18). In some embodiments, likely infection propagation paths are determined based on potentially related infections and spatially explicit propagation models.

In some embodiments, systems and method employ known information regarding different types of infections and microbial strains of infections. In some embodiments, at least some of the known information regarding different types of infections and microbial strains would be obtained from sources outside the information monitoring and tracking system. In some embodiments, at least some of the known information could be obtained from training data obtained from the healthcare system or historical information from the healthcare system. In some embodiments, at least some of the information regarding different types of infections and microbial strains could be obtained from sources outside the information monitoring and tracking system and some of the information could be obtained from training data obtained from the healthcare system or historical information from the healthcare system. In some embodiments, this information may be stored separately from a database that stores the microbial strain-based data and other types of data. An example of such information is the transmission method. For example, it is known that C. difficile can sporulate and propagate over long periods of time while MRSA requires contact, thus, the search windows for potential propagation are different (e.g., MRSA would require looking back over data corresponding to patient over a long time period and C. difficile may only require focusing on contact with patients currently in or very recently in the healthcare facility).

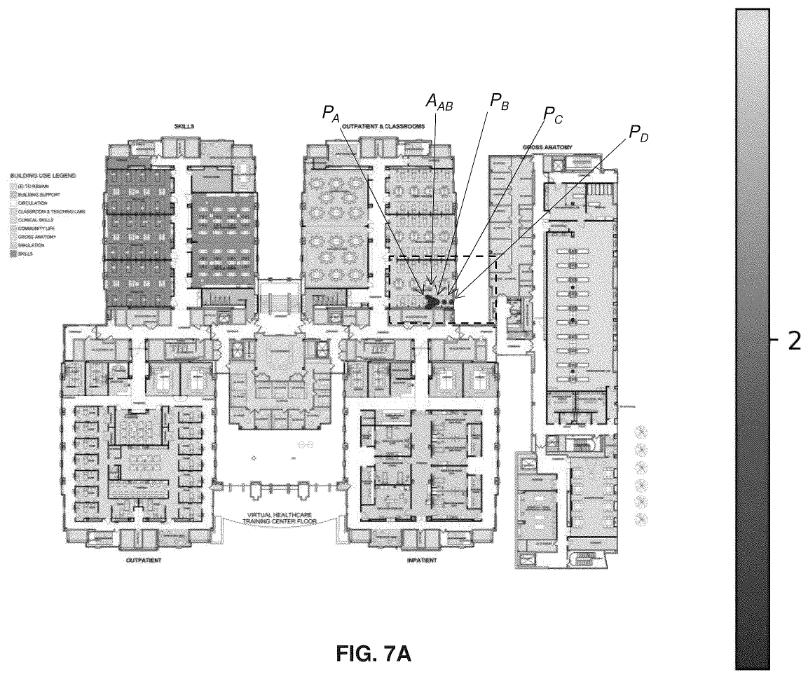

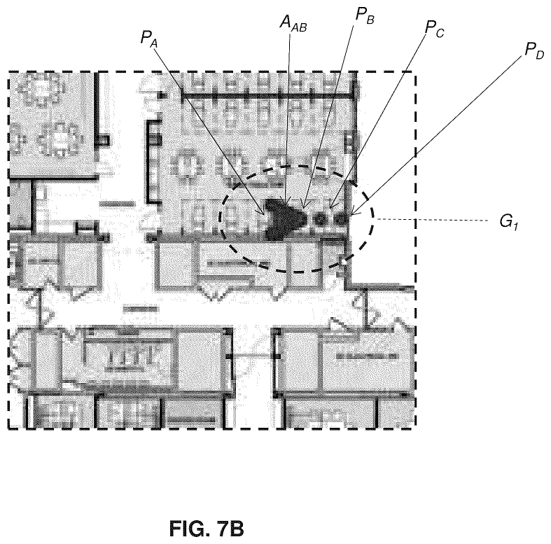

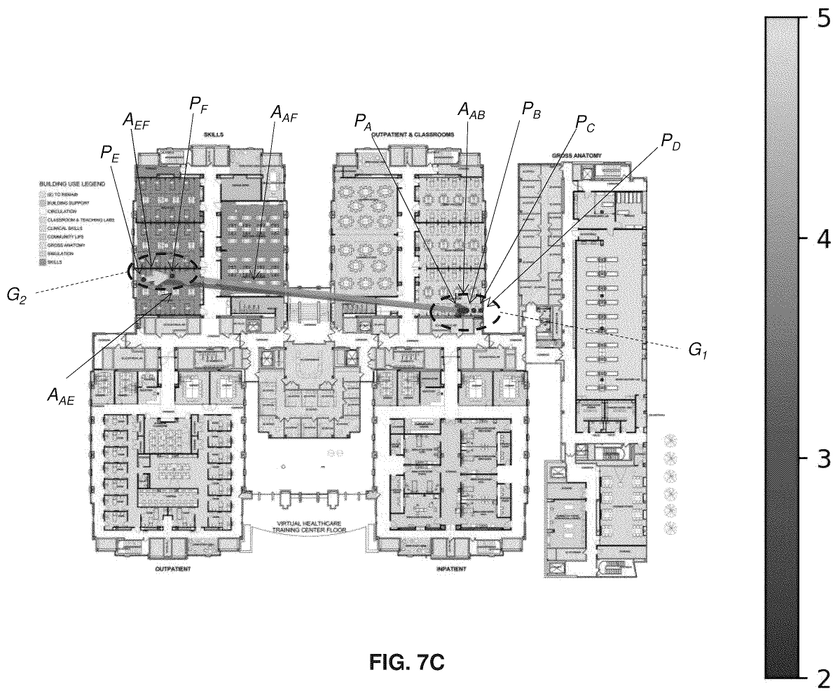

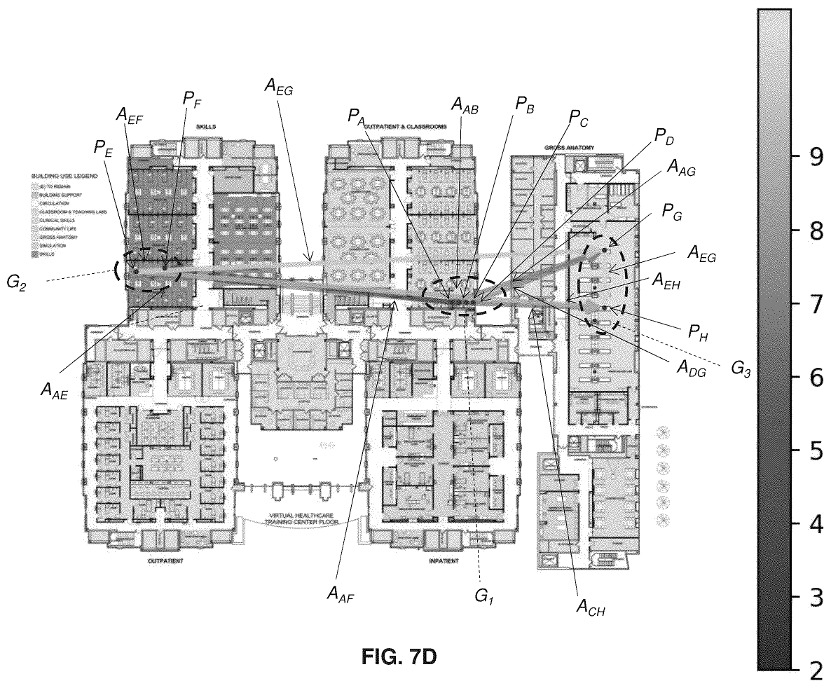

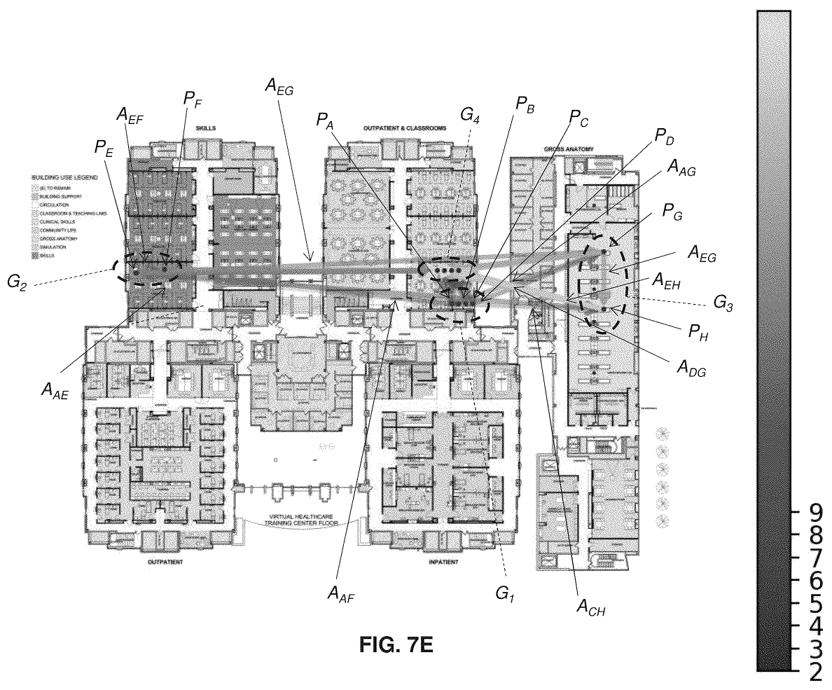

A dynamic interactive user interface for viewing infection monitoring and tracking results is generated, a report is generated on infection status, or both (see FIG. 1A, step 20). FIG. 4 schematically depicts view of a graphical user interface for viewing and exploring infection tracking results in accordance with some embodiments. The GUI may include an option for viewing floormaps graphically depicting possible transmission paths (see FIGS. 7A-7E and descriptions below). The GUI may also include an option for viewing graphs. In some graph based plots, a dynamic graphical user interface enables a user to select what connections to be displayed (e.g., based on different thresholds for a similarity function between infections such as display connections between all infections that share more than 90% similarity). The GUI may also include an option for displaying time series data (see description of FIG. 5 above). In some embodiments, the GUI may display a healthcare facility map, a map of healthcare facilities in a healthcare network system. The GUI may also include an option for displaying statistics. In some embodiments, the GUI may display large time scale propagation, mutation and frequency statistics.

In some embodiments, the GUI will include interactive visualizations to display real-time infection propagation trends, hotspots, and potential sources. In some embodiments, the GUI includes multiple information modes, and each mode can be used to display data based on any feature or a combination of features (e.g., microbial strain, type, location, department, patient, healthcare worker, etc.). In some embodiments, the GUI will also support sub queries limiting the displayed data to ranges or matches on the other categories.

In some embodiments, systems and methods may generate one or more different types of reports periodically (e.g., daily, twice a day, hourly, twice an hour, every minute), on demand, when triggered by meeting criteria, or any combination of the aforementioned. In some embodiments, one or more different types of reports are generated at least at least every day. In some embodiments, one or more different types of reports are generated at least every day, every minute, every hour, every 2 hours, every 3 hours, every 4 hours, every 5 hours, every 6 hours every 7 hours, every 8 hours, every 9 hours, every 10 hours, every 11 hours, every 12 hours, every 13 hours, every 14 hours, every 15 hours, every 16 hours, every 17 hours, every 18 hours, every 20 hours, every 21 hours, every 22 hours, every 23 hours, or every 24 hours. In some embodiments, one or more different types of reports are generated at least about once a day, about twice a day, about 3 times a day, about 4 times a day, about 5 times a day, about 6 times a day, about 7 times a day, about 8 times a day, about 9 times a day, about 10 times a day, about 11 times a day, about 12 times a day, about 13 times a day, about 14 times a day, about 15 times a day, about 16 times a day, about 17 times a day, about 18 times a day, about 19 times a day, about 20 times a day, about 21 times a day, about 22 times a day, about 23 times a day, or about 24 times a day.

For example, systems and methods may generate the same or different types of reports for hospital management and infection control teams to enable them to make informed decisions. In some embodiments, the report on infection status may be an interactive report. A user interface including a graphical user interface may enable a user to select different aspects of the report for visualization, which may be dynamic visualization.

In some embodiments, the method and system may generate interactive spatially explicit propagation views over time for the dynamic interactive user interface (see FIG. 1B, step 22 and FIGS. 7A-7E). In some embodiments, the interactive spatially explicit propagation views are generated from the spatially explicit propagation models.

For example, the user interface may display a mapping of patients with like infections (e.g., having the same microbial strain) and visually represent contacts between patients (e.g., via healthcare worker contacts and instrument contacts) over space and time (see FIGS. 7A-7E). FIG. 7A is a graphical depiction of mapping of patient infections in at least a portion of a health care facility with different colors representing areas with different types of building use and with four patients infected patients P_{A}, P_{B}, P_{C} and P_{D} having a like infection (e.g., the same microbial strain) indicated with dots. FIG. 7B is a detail of FIG. 7A including the four dots representing the four infected patients. The arrow A_{AB} from patient P_{A} to patient P_{B} indicates one or more contacts (e.g., via healthcare worker(s) and/or instrument(s)) between the patients in the relevant time period. In some embodiments, the graphical user interface may enable a user to select of a portion of the mapping and zoom in on that portion of the mapping. The relevant time period may be specific to the type of infection/and or particular microbial strain of infection. For example, the relevant time period may be based on one or more factors including, an incubation period for the infection. The scale bar to the right of the image relates the color of the arrow to number of contacts between the patients. The direction of the arrow is from P_{A} to patient P_{B} indicating that infection onset date and time was earlier for patient P_{A} than for patient P_{B}. The lack of any arrows connecting with patients P_{C} and patient P_{D} indicates that the system did not identify any contacts (e.g., via healthcare workers or via instruments) between these patients and the other patients infected with the same microbial strain. This group of initial patients that are spatially close together are referred to as a first group G₁ of patients herein. For convenience, the day depicted in FIGS. 7A and 7B will be referred to as day two of the detected infection.

FIG. 7C depicts a mapping of patient infections for the same portion of the health care facility showing patients with the same microbial strain corresponding to data for a day later that the day depicted in FIGS. 7A and 7B, which will be identified as day three of the detected infection. This mapping of infections includes a second group G₂ of two additional infected patients PE and PF who are spatially separated from the first group G₂ of infected patients (i.e., P_{A}, P_{B}, P_{C} and P_{D}). The yellow arrow A_{EF} between patients P_{E} and P_{F} indicates that according to medical records, P_{E} had the infection before P_{F} and that the system found five contacts between patient P_{E} and P_{F} (e.g., via healthcare worker(s) contacts and/or instrument(s)) in the relevant time period. This indicates that the infection could have spread from patient P_{E} to P_{F}. The purple arrow A_{AE} indicates that there were two contacts between patient P_{A} and patient P_{E} in the relevant time period, indicating that the infection could have spread from patient P_{A} to patient P_{E}. The orange arrow A_{AF} from patient P_{A} to patient P_{F} indicates that there were four contacts between patient P_{A} and patient P_{F} in the relevant time period and that the infection could have spread from patient P_{A} to patient P_{F}. In some embodiments, a graphical user interface enables a user to select an arrow and in response, the system display information about the contacts represented by the arrow (e.g., showing how many healthcare workers or instruments are represented by the arrow and information about each such as type of healthcare worker, identifier for healthcare worker, type of instrument, identifier for instrument, time or time period and date of contacts, etc.). In some embodiments, the dynamic nature of the GUI enables a user to select elements of interest, and, in response, information corresponding to that element is displayed. In some embodiments, the dynamic GUI enables a user to select an arrow, or a point on a graph, which is linked to additional information (e.g., the type or nature of a device or a healthcare worker's employee ID number).

FIG. 7D depicts a mapping of patient infections for the same portion of the health care facility showing patients infected with the same microbial strain corresponding to data for a day later that the day depicted in FIG. 7C and two days later than the day depicted in FIGS. 7A and 7B, which will be referred to herein as day four of the detected infection. This mapping of infections from day four includes a third group G₃ of two additional infected patients P_{G} and P_{H} who are spatially separated from the first group of infected patients G₁ and from the second group of infected patients G₂. The light orange arrow A_{EG} from patient P_{E} to patient P_{G} shows that there were nine contacts between patient P_{E} and patient P_{G} in the relevant time period, indicating that the infection could have spread from patient P_{E} to patient P_{G}. The yellow arrow A_{GH} between patients P_{G} and P_{H} indicates that according to medical records, P_{G} had the infection before P_{H} and that the system found more than nine contacts between patient P_{G} and P_{H} (e.g., via healthcare worker(s) contacts and/or instrument(s)) in the relevant time period. The orange arrow A_{EH} from patient P_{E} to patient P_{H} shows that there were eight contacts between patient P_{E} and patient P_{H} in the relevant time period, indicating that the infection could have spread from patient P_{E} to patient P_{H}. The light purple arrow A_{DH} from patient P_{D} to patient P_{H} shows that there were about five contacts between patient P_{D} and patient P_{H} in the relevant time period. The light purple-orange arrow A_{AG} from patient P_{A} to patient P_{G} shows that there were about six contacts between patient P_{A} and patient P_{G} in the relevant time period. The purple-orange arrow A_{DG} from patient P_{D} to patient P_{G} shows that there were about seven contacts between patient P_{A} and patient P_{G} in the relevant time period. The larger number of contacts between individual members in groups G₂ and G₃ than between groups G₁ and G₃ may indicate that it is more likely that the infection spread from group G₂ to G₃ than directly from G₁ to G₃ even though group G₁ is spatially closer to group G₃.

FIG. 7E depicts a mapping of patient infections for the same portion of the health care facility showing patients infected with the same microbial strain corresponding to data for three days later than the day depicted in FIGS. 7A and 7B, which will be referred to herein as day five of the detected infection. This mapping of infections from day five includes a fourth group G₄ of four additional infected patients that are spatially relatively close to the first group of infected patients G₁, but spatially separated from and further from the second G₂ and third G₂ groups of infected patients. As depicted by the arrows, the fourth group of infected patients G₄ has contacts with all the other groups of infected patients indicating possible spread from any of the other groups, but has the most contacts with the third group G₄ of infected patients indicating a higher relative likelihood that the infection spread from the third group G₃ and specifically with a contact with P_{H}.

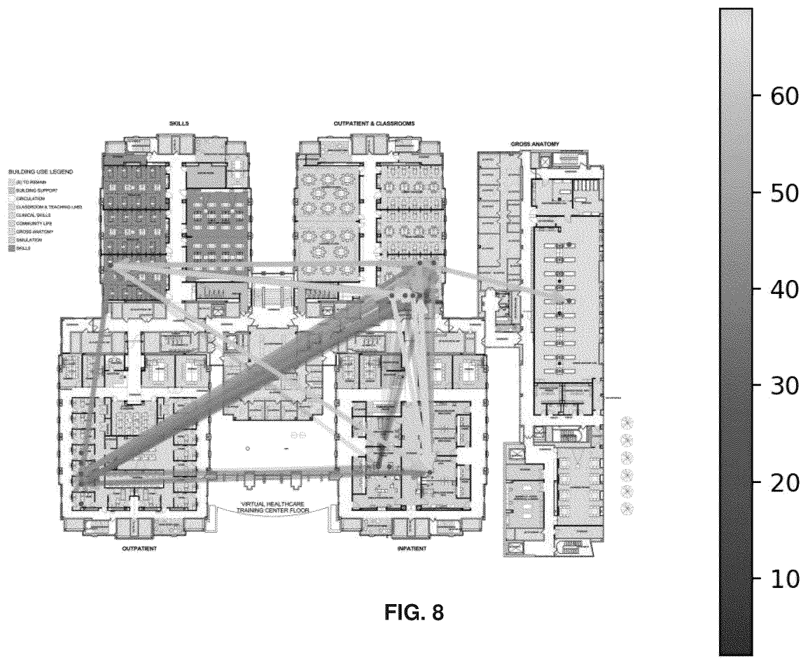

FIG. 8 depicts another example of a map of patient infections for a different outbreak to be displayed in a GUI with more infected patients and more contacts than those appearing in FIGS. 7A-7C, in accordance with some embodiments. In some embodiments, the graphical user interface may include dynamic images or animations tracing possible paths of infection backward and/or forward in time. In some embodiments, patients are mapped onto floorplans, and displayed as an animation over time, with contacts displayed as arrows. In some embodiments, the graphical user interface may have a user selectable element to enable the display go forward and backward in time with respect to the data displayed based on the user selection.

In some embodiments, mapping patients and healthcare workers (and in some embodiments also instruments) in time and space enables efficient identification of whether two patients with the same infectious species (e.g., two patients with MRSA) that overlap in time at the same hospital, can be linked by having been contacted by the same healthcare worker or the same instrument. In some embodiments, the system's identification of which patient was contacted first (e.g., via the direction of the arrow) aids in efficient analysis for the identification of possible source(s) for the infection. Significantly, by providing an accurate and timely history of healthcare worker and instrument contacts with infectious patients, systems and methods may shorten the time required to identify potential sources of infection (e.g., patients, healthcare workers, and instruments) enabling faster action to reduce the likelihood or extent of spread of the infection. As an example, for MRSA infections, if there is suspicion of transmissions by healthcare worker, the healthcare worker can be tested to determine whether he or she is a carrier and the potential source.

The GUI may dynamically visualize various aspects of infection tracking analysis or results or of the report and enable a user to select different aspects of the analysis or report to explore details. The systems and methods will identify suspected transmission events by mapping patients with like infections and evaluating their movement and healthcare contacts over time and space. The GUI may employ visualization and animation to aid a user in exploring and understanding transmission paths.

In some embodiments, the GUI may include similarity displays. For example the GUI may include an antibiogram with multiple microbial strains superimposed (e.g., see, FIG. 6C) with distance statistics. When other variant determining data becomes more readily available (e.g., genotyping) distance metrics could be replaced by sequence distance metrics. In some embodiments, the GUI may include sequence trees and sequence alignment-based displays for genetic information from different microbial strains or infection populations (e.g., single nucleotide polymorphisms (SNPs), homology, and annotations).

In some embodiments, the infection tracking and monitoring methods and systems will periodically or continuously update the separate database with new interactions and new infection cases.

Some embodiments employ machine learning models. For example, regression analysis may be employed for one or more of feature association, risk evaluation, or outbreak detection. Other types of machine learning models that may be employed (e.g., for feature association, risk evaluation, or outbreak detection) include Support Vector Machines (SVM), Random Forest, Gradient Boosting Machines (GBM), Artificial Neural Networks (ANN), Gaussian Processes, etc. In some embodiments, Bayesian methods may be employed for propagation model parameterization. In some embodiments, K-means clustering may be employed for microbial strain thresholding. In some embodiments, a large language model (LLM) may be employed in the user interface and/or query design.

In some embodiments, the infection tracking and monitoring methods and systems will include an alert system or a notification system or alert or notification features to alert or notify healthcare personnel about potential infection clusters or unusual patterns. In some embodiments, the systems and methods employ thresholds and/or triggers for the early detection of infection propagation.

In some embodiments, the systems and methods generate one or more of a rapid increase alert, an active propagator alert, or an emerging pathogen alert, as described below (FIG. 1B, step 24).

In some embodiments, systems and methods may include a rapid increase alert or notification. In some embodiments, a rapid increase alert or notification may be issued whenever a per feature category rate of increase for a given pathogen goes beyond a threshold based on previous data.

In some embodiments, systems and methods may generating include an active propagator alert or notification. In some embodiments, an active propagator alert or notification may be issued based on a contact feature category (e.g., a healthcare worker, instrument) being identified to have been in contact with more than a statistically expected number of patients with the same microbial strain of infection in a timeframe.

In some embodiments, systems and methods may include an emerging pathogen alert or notification. An emerging pathogen alert or notification may be issued based on one or more of: 1) appearance of a pathogen having a combination of resistances that has not been previously observed in known pathogens; 2) detection of an infection with an increase in resistance to a drug larger than a determination error for the drug; 3) detection of a non-cultivable pathogen not matching known microbial strains; 4) detection of a pathogen with a genotype not matching previous known data (e.g., by multilocus sequence typing (MLST)); 5) detection of a pathogen not matching previous known data by genomic sequence (e.g., using appropriate thresholds for homology based on previous per species data); and 6) detection of a pathogen not matching previous known data as detected by any suitable genomic-based technology.

In some embodiments, the criteria for an emerging pathogen alert may be automatically set. In some embodiments, thresholding for detection criteria for an emerging pathogen alert may be specific to each pathogen. In some embodiments, thresholding for detection criteria for an emerging pathogen alert may be based on previously collected data and machine learning methods producing parameters specific to each pathogen.

In some embodiments, alerts and notifications will include at least some or all of information on the microbial strain, characteristics of the pathogen, and locations and personnel affected.

In some embodiments, a standard report may be issued with rankings for each feature categories (e.g., healthcare worker most likely to be propagating X pathogen, instrument most likely to be propagating X pathogen).

In some embodiments, alerts and/and notification may appear in a GUI associated with the infection tracking and monitoring system upon a user opening or accessing the GUI associated with the infection tracking and monitoring system. In some embodiments, alerts and notifications may be pushed to a computing device or mobile device of a user (e.g., via text, via phone call, via popup notification). In some embodiments, the content of the alert or notification may be pushed to a user. In some embodiments, a notification of a type of alert or notification may be pushed to a user (e.g., type of alert and link to alert) and the user accesses a GUI of the system to view the content of the alert or notification.

In some embodiments, the method or system may generate one or both of a pathogen propagators report or a key propagators report (FIG. 1B, step 26).

In some embodiments, the method or system may generate a recommended list of infections for molecular analysis (FIG. 1B, step 28). In some embodiments, infections may be prioritized for molecular analysis based on one or more of an increase from expected number of cases above threshold, a new antibiotic resistance, or a new variant on MLSTs.

In some embodiments, the system or method may generate a prioritized list of infections for follow up or further analysis. In some embodiments, the list may be prioritized based on one or more of an increase from expected number of cases above threshold, a new antibiotic resistance, or an identification of an emerging variant.

In some embodiments, the infection monitoring and tracking systems and methods employ machine learning models (e.g., network analysis, time-series analysis, and probabilistic models) to detect infection propagation patterns. In some embodiments, machine-learning models may be trained on historical data to learn dynamics of infection spread within a particular healthcare facility or healthcare system. In some embodiments, a model for infection propagation may be updated or fine-tuned as new data becomes available.

In some embodiments, use of the dynamic infection monitoring and tracking systems and methods described herein may facilitate improved strategies for rapid containment and isolation of infected individuals and areas. In some embodiments, use of the dynamic infection monitoring and tracking systems and methods described herein may enable implementation of automated containment protocols based on the severity and extent of infection spread.

Dynamic infection monitoring and tracking systems and methods herein are not limited to a single healthcare facility or to patients who are inpatient. In some embodiments, patients, healthcare workers, instruments, and infections may be tracked throughout a healthcare system, whose facilities could be contiguous or geographically separated. In some embodiments, patients may be tracked while they are inpatient and also while outpatient (e.g., for follow up appointments, etc.) for infection tracking. In some embodiments, patients who are solely outpatient (e.g., some chemotherapy treatment patients, some radiation therapy patients, some dialysis patients, patients in the ER who are not admitted) and instruments used on those patients may also be tracked. In some embodiments, generation of an alert or a notification may be based on at least some criteria that reflect infections affecting more than one healthcare facility. For example, one criteria may be a threshold for a rise in a type or microbial strain of infection across multiple different healthcare facilities in a certain period of time. In some embodiments, analysis of infections across multiple different facilities that may be graphically separate may enable early detection of a widespread event (e.g., a beginning of a pandemic or an imminent health threat).

In some embodiments, genotyping of microbial infection strains may be widely applied such that antibiograms are of less importance or are not employed. For example, genotyping of microbial infection strains may become more affordable and have a faster turnaround such that antibiograms are not needed. Embodiments described herein may employ genotyping of microbial infection strains and/or antibiograms for microbial infection strains and/or any other suitable techniques or methods for determining relationships between different populations of infections.

The dynamic infection propagation detection system and methods described herein will provide healthcare facilities and systems with powerful tools to proactively manage and contain infections. By leveraging data on patient, healthcare worker, and instrument interactions, the systems and methods enhance patient safety and reduce the risk of infection outbreaks in healthcare settings.

### Computing Systems, Networks and Devices for Implementing Some Embodiments

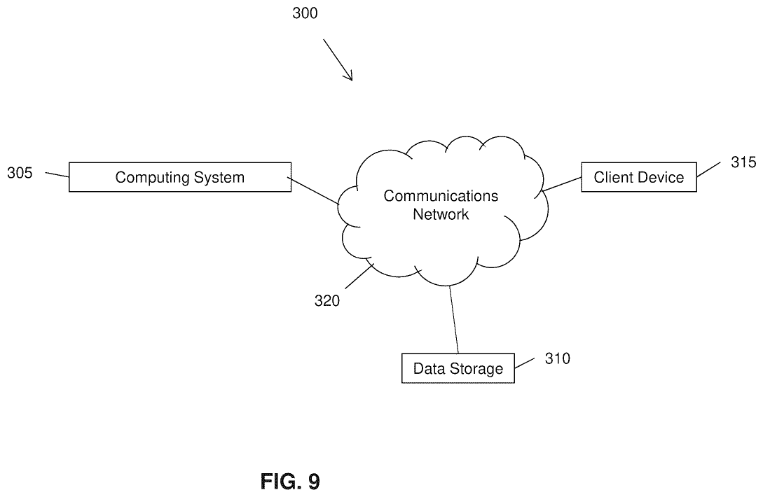

FIG. 9 schematically depicts a network 300, alternately described as a networked computing system, for implementing some aspects in accordance with some embodiments. Network 300 may include at least one computing system 305, at least one client device 315, and data storage 310 that may be in the form of one or more databases. In some embodiments, computing system 305, client device 315, readout device 317, and/or data storage 310 may be connected to network 320. However, in other embodiments, two or more of computing system 305, client device 315, and/or data storage 310 may be connected directly with each other, without network 320. While one computing system 305, one client device 315, and one data storage 310 are shown in FIG. 9, it should be appreciated that any number of computing systems, client devices, and data storages could be used.

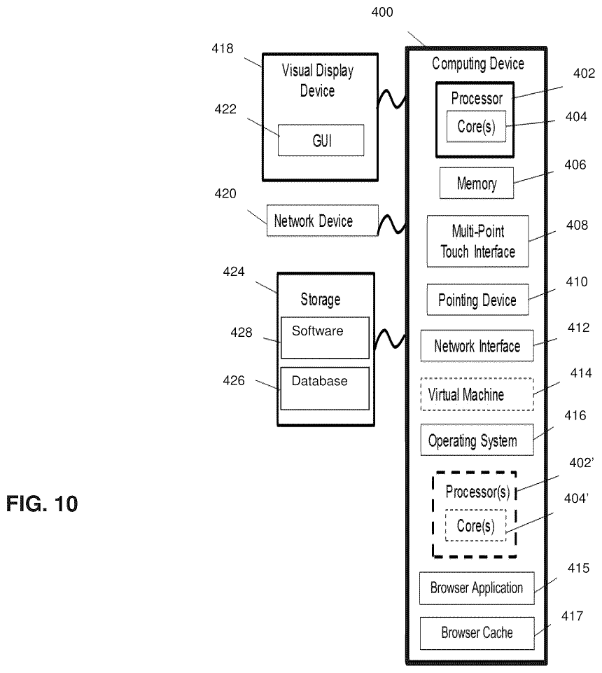

Computing system 305 may include one or more computing devices configured to perform one or more operations consistent with disclosed embodiments. Computing system 305 is further described in connection with FIG. 10. In some embodiments, computing system 305 may perform at least some aspects or steps of the described methods. In some embodiments, computing system 305, and/or client device 315 may perform at least some aspects or steps of the described methods in some embodiments. For example, in some embodiments, client device 315 may be used to present a graphical user interface and may include an “app” for communication with a remote computing system.

Data storage 310 may include one or more computing devices configured with appropriate software to perform operations consistent with storing and providing data. Data storage 305 may include, for example, Oracle™ databases, Sybase™ databases, or other relational databases or non-relational databases, such as Hadoop™ sequence files, HBase™, or Cassandra™ Data storage 310 may include computing components (e.g., database management system, database server, etc.) configured to receive and process requests for data stored in memory devices of data storage 310 and to provide data from data storage 310. In some embodiments, data storage 305 may be configured to store current information from EMRs or an EMR system or any other data required by or produced by computing system 305 or client device 315. While data storage 310 is shown separately, in some embodiments, data storage 310 may be included in or otherwise related to computing system 305 and/or client device 315.

Client device 315 may include a desktop computer, a laptop, a server, a mobile device (e.g., tablet, smart phone, etc.), a wearable computing device, or other type of computing device. Client device 315 may include one or more processors configured to execute software instructions stored in memory, such as memory included in client device 315. In some embodiments, client device 315 may include software that when executed by a processor performs known Internet-related communication and content display processes. For instance, client device 315 may execute browser software that generates and displays interfaces including content on a display device included in, or connected to, client device 315. Client device 315 may execute applications that allows client device 315 to communicate with components over network 370 and generate and display content in interfaces via display devices included in client device 315. For example, client device 315 may display results produced by computing system 305, such as graphs, images etc. Computing system 305 may communicate results of analysis or reports to the client device 315.

Computing system 305, client device 315, and database 315 are shown as different components. However, computing system 305, client device 315, and/or database 315 may be implemented in the same computing system or device. For example, computing system 305, client device 315, and/or database 315 may be embodied in a single computing device. In some embodiments various functions or features could be implemented in a distributed manner or using a cloud computing or storage system instead of or along with computing system 305, data storage 310 and client device 315. In some embodiments, client device 315 may have an app installed that provides the GUI.

Network 320 may be any type of network configured to provide communications between components of network 320. For example, network 320 may be any type of network (including infrastructure) that provides communications, exchanges information, and/or facilitates the exchange of information, such as the Internet, a Local Area Network, near field communication (NFC), optical code scanner, or other suitable connection(s) that enables the sending and receiving of information between the components of network 320. In other embodiments, one or more components of network 320 may communicate directly through a dedicated communication link(s).

FIG. 10 schematically depicts a computing system 400 for implementing some aspects in accordance with some embodiments. In some embodiments, computing device 400 may be computing system 305 shown in FIG. 10. In some embodiments, computing device 400 may be client device 315 shown in FIG. 10. Computing device 400 includes one or more non-transitory computer-readable media for storing one or more computer-executable instructions or software for implementing exemplary embodiments. The non-transitory computer-readable media can include, but are not limited to, one or more types of hardware memory, non-transitory tangible media (for example, one or more magnetic storage disks, one or more optical disks, one or more USB flash drives), and the like. For example, memory 406 included in the computing device 400 can store computer-readable and computer-executable instructions or software for implementing exemplary embodiments. Computing device 400 also includes processor 402 and associated core 404, and optionally, one or more additional processor(s) 402′ and associated core(s) 404′ (for example, in the case of computer systems having multiple processors/cores), for executing computer-readable and computer-executable instructions or software stored in the memory 406 and other programs for controlling system hardware. Processor 402 and processor(s) 402′ can each be a single core processor or multiple core (404 and 404′) processor.

Virtualization can be employed in computing device 400 so that infrastructure and resources in the computing device can be shared dynamically. A virtual machine 414 can be provided to handle a process running on multiple processors so that the process appears to be using only one computing resource rather than multiple computing resources. Multiple virtual machines can also be used with one processor.

Memory 406 can include a computer system memory or random-access memory, such as DRAM, SRAM, EDO RAM, and the like. Memory 406 can include other types of memory as well, or combinations thereof. An individual can interact with the computing device 400 through a visual display device/graphical user interface (GUI) 418, such as a touch screen display or computer monitor, which can display one or more user interfaces 422 for displaying data to the individual. The visual display device 418 can also display other aspects, elements and/or information or data associated with exemplary embodiments. The computing device 400 can include other input devices and I/O devices for receiving input from an individual, for example, a keyboard, a scanner, or another suitable multi-point touch interface 408, a pointing device 410 (e.g., a pen, stylus, mouse, or trackpad). The keyboard 408 and the pointing device 410 can be coupled to the visual display device 418. The computing device 400 can include other suitable conventional I/O peripherals.

The computing device 400 can also include one or more storage devices 424, such as a hard-drive, CD-ROM, or other computer readable media, for storing data and computer-readable instructions and/or software that implement exemplary embodiments of the system as described herein, or portions thereof. Exemplary storage device 424 can also store one or more databases for storing suitable information required to implement exemplary embodiments. The databases can be updated by an individual or automatically at a suitable time to add, delete or update data in the databases. Exemplary storage device 424 can store datasets 426, software 428, and other data/information used to implement exemplary embodiments of the systems and methods described herein. The computing device 400 can include a network interface 412 configured to interface via one or more network devices 420 with one or more networks, for example, Local Area Network (LAN), Wide Area Network (WAN) or the Internet through a variety of connections including, but not limited to, standard telephone lines, LAN or WAN links (for example, 802.11, T1, T3, 56kb, X.25), broadband connections (for example, ISDN, Frame Relay, ATM), wireless connections, processing device area network (CAN), or some combination of any or all of the above. The network interface 412 can include a built-in network adapter, network interface card, PCMCIA network card, card bus network adapter, wireless network adapter, USB network adapter, modem or another device suitable for interfacing the computing device 400 to a type of network capable of communication and performing the operations described herein. Moreover, the computing device 400 can be a computer system, such as a workstation, desktop computer, server, laptop, handheld computer, tablet computer (e.g., the iPad® tablet computer), mobile computing or communication device (e.g., the iPhone® communication device), or other form of computing or telecommunications device that is capable of communication and that has sufficient processor power and memory capacity to perform the operations described herein.

The computing device 400 can run an operating system 416, such as versions of the Microsoft® Windows® operating systems, the different releases of the Unix and Linux operating systems, a version of the MacOS® for Macintosh computers, an embedded operating system, a real-time operating system, an open source operating system, a proprietary operating system, an operating systems for mobile computing devices, or another operating system capable of running on the computing device and performing the operations described herein. In exemplary embodiments, the operating system 416 can be run in native mode or emulated mode. In an exemplary embodiment, the operating system 416 can be run on one or more cloud machine instances.

The present invention may be embodied within a system, a method, a computer program product or any combination thereof. The computer program product may include a computer readable storage medium or media having computer readable program instructions thereon for causing a processor to carry out aspects of the present invention. The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing.

Computer readable program instructions described herein can be downloaded to the respective computing/processing devices from a computer readable storage medium or to an external computer or external storage device via a network. The computer readable program instructions may execute entirely on the user's computer, partly on the user's computer, as a stand-alone software package, partly on the user's computer and partly on a remote computer or entirely on the remote computer or server. Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein includes an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which includes one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

### Example

An example dynamic infection monitoring and tracking system was built and tested using anonymized healthcare data from seventeen hospitals in the Hackensack Meridian Health System regarding infections over a period from early December 2023 to January 2024. The example system, which was updated with new data daily, monitored infection data, determined which infections were likely related, tracked contacts between patients, health workers, and instruments based on documented contacts as well as contacts based on overlap in geographical space and time, and determined likely infection paths. The system also generated dynamic graphical representations of contacts and path tracing to aid infection control practitioners.

### Data Retrieval

Data was posted from an EMR system, specifically, the EPIC system, and a laboratory information system, specifically Beaker Laboratory Information System by EPIC Systems Corp., continuously onto a spreadsheet. The system included an application that pulled in the data on changes and updated a local database (e.g., an IMaT database) daily. The data contained in the local database included patient data, healthcare worker data, instrument data, and infection data obtained from the EPIC EHR system and the Beaker Laboratory Information System.

Patient data was retrieved for fields including: last date refreshed, patient ID, patient medical record number ID, infection ID, infection onset date and time, infection onset date, local date and time, specimen source, specimen type, infection type, infection onset year and month, patient encounter contact serial number ID, administrative discharge department ID, administrative discharge department, location ID, administrative discharge location, hospital admission date and time, hospital discharge date and time, contact date, discharge department ID, administrative department ID, not yet discharged, admission type, discharge deceased (boolean), bed ID, room number, label for bed, treatment team, surgical log ID, surgical case ID, surgical procedure date, surgery start date and time, surgery end date and time, inpatient (Y/N), surgery primary procedure code, surgery procedure name, surgery primary physician, surgery secondary physician, surgical patient contact serial number, surgical case class, surgical patient class, surgical equipment ID, surgical department ID, and surgical department.

Retrieved infection data included the following fields: patient ID, patient medical record number ID, patient encounter contact serial number ID, infection ID, infection onset date and time, specimen source, specimen type, infection type, discharged deceased (boolean), administrative discharge department, test type requested, order contact date and time, procedure ID, order description, order authorization provider ID, specimen taken date and time, abnormal (Y/N), organism, result date, antibiotic LOINC code, antibiotic susceptibility, source of antibiotic LOINC code, sensitivity to antibiotic value, lab name, and refreshed date

Infection information or infection data (e.g., plasmids, mlsts, antibiogram) can be used to build a vector of potentially relevant features, which are those features for which there is a variation for species that could define the relevant features of that species. Infection data was converted to a per infection object, which contained the metadata and a vector representation, where each dimension was the resistance to a specific drug.

Patient data was converted to a set of directed graph type object, where contacts were determined by shared metadata (locations, instruments, healthcare workers, etc.) and likelihood of a common infection derived from the infection-based data.

### Data Integration and Preprocessing

The collected data was merged and cleaned to create a comprehensive dataset. The comprehensive dataset was normalize and standardized for consistency and accuracy. Missing data was handled through imputation techniques.

Features were created such as interaction frequency, contact duration, and proximity to potential infection sources.

The system-generated images of time series of infection frequencies (see FIG. 5). The system analyzed antibiograms of infected cell populations and determined the likelihood that various infections were related (see FIG. 6C) The system also determined likely infection spread paths based on analysis of microbial infection strain using spatially implicit models, and analysis of contacts using spatially explicit models. The system also generated dynamic depictions of geographical path tracing for infections, which are referred to herein as propagation views (see FIGS. 7A-7D).

The example system demonstrated that power and effectiveness of a dynamic infection tracking and monitoring system that could track infections on a daily basis or on a shorter time period using both documented contacts between patient, healthcare workers, and instruments and using inferred contacts based on overlap in space and time. Such a system enables infection control practitioners to identify infections of particular interest and infections that could potentially start an outbreak earlier in time. Such a system also facilitates early and efficient identification of propagators of disease, whether they are patients, healthcare workers, or instruments.

Finally, the terminology used herein is for describing particular embodiments only and is not intended to be limiting of the invention. As used herein, the singular forms “a”, “an” and “the” are intended to include the plural forms as well, unless the context clearly indicates otherwise. It will be further understood that the terms “includes” and/or “including,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

1. A method for infection tracking and monitoring in a healthcare facility or a healthcare system, the method comprising:
accessing or receiving current information from electronic medical records or an electronic medical records system and a laboratory information system, the current information comprising: patient data, healthcare worker data, instrument data, and infection data; the patient data including, for each patient identified as having an infection: an identifier for the patient, demographic information, medical history, and admission information where admitted; the healthcare worker data including, for each healthcare worker: an identifier for the healthcare worker, a role or position of the healthcare worker; the instrument data including for each instrument: an identifier for the instrument and record information about the usage of the instrument; and the infection data including: cases of infections among patients including, for each case: type of infection, and date of diagnosis;
storing the current information in an infection monitoring and tracking (IMaT) database;
accessing, receiving, or generating additional current information including updated patient data, updated healthcare worker data, updated instrument data, and updated infection data periodically or on demand and storing the additional current information in the IMaT database, the current information and the additional current information identified herein as collected information;
determining interaction data for each patient from the collected information based on documented interactions between the patient and one or more healthcare workers, based on documented uses of one or more instruments on the patient, and based on spatial location, date, and time overlap between the patient and the one or more healthcare workers and storing the determined interaction data in the IMaT database;
identifying potentially related infections based, at least in part, on the infection data;
determining one or more likely infection propagation paths based, at least in part, on the identified potentially related infections and the interaction data; and
generating a dynamic interactive user interface enabling a user to view and interact with visualizations of infection monitoring and tracking results and/or generating a report on infection status including infection monitoring and tracking results where the infection monitoring and tracking results include the determined one or more likely infection propagation paths.

2. The method of claim 1, wherein the infection data further comprises microbial strain characterization of the infection for at least some of the infections.

3. The method of claim 2, wherein the microbial strain characterization of infection data includes antibiogram data, genome sequencing data, or both for at least some of the infections.

4. The method of any one of claims 1-3, further comprising generating a spatially explicit infection propagation view depicting spatial relationships between infected patients and a structure of at least a portion of a healthcare facility for display in the dynamic interactive user interface based on the determined likely propagation paths.

5. The method of claim 4, wherein the spatially explicit infection propagation view includes graphical representations of contacts between individual infected patients identified as having a potentially related infection.

6. The method of claim 5, wherein the dynamic interactive user interface is configured to receive a selection of a graphical representation of contacts between two individual infected patients identified as having a potentially related infection from a user and to display details regarding the contact based on receiving the selection.

7. The method of claim 5 or claim 6, wherein the dynamic interactive user interface is configured to receive input from a user regarding at least one parameter for identifying potentially related infections based on the infection data and to display a resulting change to the graphical representations of contacts and/or to the patients identified as having potentially related infections.

8. The method of any one of claims 1-7, wherein the collected data is preprocessed, normalized, or both prior to being stored in the IMaT database.

9. The method of any one of claims 1-7, wherein the collected data stored in the IMaT database is analyzed to generate enriched data and wherein the enriched data includes at least some of the interaction data.

10. The method of any one of claims 1-9, wherein the method includes generating one or more spatially implicit models based, at least in part, on the infection data and the patient data.

11. The method of claim 10, wherein identifying potentially related infections includes performing a clustering analysis based on one of the spatially implicit models.

12. The method of any one of claims 1-11, wherein the method includes generating one or more spatially explicit models based, at least in part, on patient data, interaction data, and the identified potentially related infections.

13. The method of claim 12, wherein the likely propagation paths are determined based, at least in part, on the potentially related infections and at least one of the one or more spatially explicit models.

14. The method of any one of claims 1-13, further comprising generating or issuing an alert or notification.

15. The method of claim 14, wherein the alert or notification is a rapid increase alert or notification.

16. The method of claim 15, wherein issuance of the rapid increase alert or notification is based on pathogen-specific detection criteria.

17. The method of claim 16, wherein the pathogen-specific detection criteria is based on a rate of increase for a specific pathogen being greater than a pathogen-specific threshold based on prior data for the pathogen.

18. The method of claim 17, wherein the pathogen-specific threshold is based on prior data for the pathogen.

19. The methods of claim 17, wherein the pathogen-specific threshold is based on prior data for infections for the pathogen at the healthcare facility or healthcare system.

20. The method of claim 14, wherein the alert or notification is an active propagator alert or notification.

21. The method of claim 20, wherein the active propagator alert or notification is issued based on a healthcare worker or an instrument being identified to have been in contact with more than a statistically expected number of patients with potentially related infections in a timeframe.

22. The method of claim 14, wherein the alert or notification is an emerging pathogen alert or notification.

23. The method of claim 22, wherein issuance of the emerging pathogen alert or notification is based on one or more of:
detection of the appearance of a pathogen having a combination of resistances that has not been previously observed in known pathogens;
detection of an infection with an increase in resistance to a drug larger than a determination error for the drug;
detection of a non-cultivable pathogen not matching known microbial strains;
detection of a pathogen with a genotype not matching previous known data is detected;
detection of a pathogen not matching previous known data as determined by genomic sequencing; or
detection of a pathogen not matching previous known data as detected by a genomic-based technology.

24. The method of any one of claims 14 to 23, wherein the alert or notification includes at least some or all of information on a microbial strain of infection, characteristics of a pathogen for the infections, a location involved, personnel involved, or an instrument involved.

25. The method of any one of claims 1-24, further comprising:
generating a pathogen propagator's report; or
generating a key propagators report.

26. The method of any one of claims 1-25, further comprising generating a recommended list of one or more infections for molecular analysis.

27. The method of any one of claims 1-26, further comprising identifying a one or more infections as a priority for analysis and generating or transmitting an order for a molecular analysis of the one or more infections.

28. The method of any one of claim 1-27, wherein updated patient data, updated healthcare worker data, updated instrument data, and updated infection data are obtained periodically or on demand at least on a daily basis or more frequently than a daily basis; and
wherein the determining of interaction data for each patient occurs on a daily basis or more frequently than a daily basis.

29. A non-transitory computer readable medium comprising instructions that, when executed by a computing system including one or more processors, cause the computing system to perform the method of any one of claims 1 to 28.

30. A system for infection tracking and monitoring for a healthcare facility or a healthcare system, the system including:
at least one database including an infection monitoring and tracking (IMaT) database; and
one or more processors configured to execute instructions that, when executed by the one or more processors, cause the computing system to:
access or receive current information from electronic medical records or an electronic medical records system and a laboratory information system, the current information comprising: patient data, healthcare worker data, instrument data, and infection data; the patient data including, for each patient identified as having an infection: an identifier for the patient, demographic information, medical history, and admission information where admitted; the healthcare worker data including, for each healthcare worker: an identifier for the healthcare worker, a role or position of the healthcare worker; the instrument data including for each instrument: an identifier for the instrument and record information about the usage of the instrument; and the infection data including: cases of infections among patients including, for each case: type of infection, and date of diagnosis;
store the current information in the IMaT database;
access, receive, or generate additional current information including updated patient data, updated healthcare worker data, updated instrument data, and updated infection data periodically or on demand and storing the additional current information in the IMaT database, the current information and the additional current information identified herein as collected information;
determine interaction data for each patient from the collected information based on documented interactions between the patient and one or more healthcare workers, based on documented uses of one or more instruments on the patient, and based on spatial location, date, and time overlap between the patient and the one or more healthcare workers and storing the determined interaction data in the IMaT database;
identify potentially related infections based, at least in part, on the infection data;
determine one or more likely infection propagation paths based, at least in part, on the identified potentially related infections and the interaction data; and
generate a dynamic interactive user interface enabling a user to view and interact with visualizations of infection monitoring and tracking results and/or generating a report on infection status including infection monitoring and tracking results where the infection monitoring and tracking results include the determined one or more likely infection propagation paths.

31. The system for infection tracking and monitoring of claim 30, wherein the infection data further comprises microbial strain characterization of the infection for at least some of the infections.

32. The system for infection tracking and monitoring of claim 31, wherein the microbial strain characterization of infection data includes antibiogram data, genome sequencing data, or both for at least some of the infections.

33. The system for infection tracking and monitoring of claim 31 or claim 32, wherein the instructions, when executed by the one or more processors, further cause the computing system to generate a spatially explicit infection propagation view depicting spatial relationships between infected patients and a structure of at least a portion of a healthcare facility for display in the dynamic interactive user interface based on the determined likely propagation paths.

34. The system for infection tracking and monitoring of any one of claims claims 31 to 33, wherein the spatially explicit infection propagation view includes graphical representations of contacts between individual infected patients identified as having a potentially related infection.

35. The system for infection tracking and monitoring of claim 34, wherein the dynamic interactive user interface is configured to receive a selection of a graphical representation of contacts between two individual infected patients identified as having a potentially related infection from a user and to display details regarding the contact based on receiving the selection.

36. The system for infection tracking and monitoring of claim 34 or claim 35, wherein the dynamic interactive user interface is configured to receive input from a user regarding at least one parameter for identifying potentially related infections based on the infection data and to display a resulting change to the graphical representations of contacts and/or to the patients identified as having potentially related infections.

37. The system for infection tracking and monitoring of any one of claims 30-36, wherein the collected data is preprocessed, normalized, or both prior to being stored in the IMaT database.

38. The system for infection tracking and monitoring of any one of claims 30-37, wherein the collected data stored in the IMaT database is analyzed to generate enriched data and wherein the enriched data includes at least some of the interaction data.

39. The system for infection tracking and monitoring of any one of claims 30-38, wherein the instructions, when executed by the one or more processors, cause the computing system to generate one or more spatially implicit models based, at least in part, on the infection data and the patient data.

40. The system for infection tracking and monitoring of claim 39, wherein the identification of potentially related infections includes performing a clustering analysis based on one of the spatially implicit models.

41. The system for infection tracking and monitoring of any one of claims 30-40, wherein the instructions, when executed by the one or more processors, cause the computing system to generate one or more spatially explicit models based, at least in part, on patient data, interaction data, and the identified potentially related infections.

42. The system for infection tracking and monitoring of claim 41, wherein the likely propagation paths are determined based, at least in part, on the potentially related infections and at least one of the one or more spatially explicit models.

43. The system for infection tracking and monitoring of any one of claims 30-42, wherein the instructions, when executed by the one or more processors, cause the computing system to generate or issue an alert or notification.

44. The system for infection tracking and monitoring of claim 43, wherein the alert or notification is a rapid increase alert or notification.

45. The system for infection tracking and monitoring of claim 44, wherein issuance of the rapid increase alert or notification is based on pathogen-specific detection criteria.

46. The system for infection tracking and monitoring of claim 45, wherein the pathogen-specific detection criteria is based on a rate of increase for a specific pathogen being greater than a pathogen-specific threshold based on prior data for the pathogen.

47. The system for infection tracking and monitoring of claim 46, wherein the pathogen-specific threshold is based on prior data for the pathogen.

48. The system for infection tracking and monitoring of claim 46, wherein the pathogen-specific threshold is based on prior data for infections for the pathogen at the healthcare facility or healthcare system.

49. The system for infection tracking and monitoring of claim 43, wherein the alert or notification is an active propagator alert or notification.

50. The system for infection tracking and monitoring of claim 49, wherein the active propagator alert or notification is issued based on a healthcare worker or an instrument being identified to have been in contact with more than a statistically expected number of patients with potentially related infections in a timeframe.

51. The system for infection tracking and monitoring of claim 50, wherein the alert or notification is an emerging pathogen alert or notification.

52. The system for infection tracking and monitoring of claim 51, wherein issuance of the emerging pathogen alert or notification is based on one or more of:
detection of the appearance of a pathogen having a combination of resistances that has not been previously observed in known pathogens;
detection of an infection with an increase in resistance to a drug larger than a determination error for the drug;
detection of a non-cultivable pathogen not matching known microbial strains;
detection of a pathogen with a genotype not matching previous known data is detected;
detection of a pathogen not matching previous known data as determined by genomic sequencing; or
detection of a pathogen not matching previous known data as detected by a genomic-based technology.

53. The system for infection tracking and monitoring of any one of claims 43-52, wherein the alert or notification includes at least some or all of information on a microbial strain of infection, characteristics of a pathogen for the infections, a location involved, personnel involved, or an instrument involved.

54. The system for infection tracking and monitoring of any one of claims 30-53, wherein the instructions, when executed by the one or more processors, further cause the computing system to:
generate a pathogen propagator's report; or
generate a key propagators report.

55. The system for infection tracking and monitoring of any one of claims 30-54, wherein the instructions, when executed by the one or more processors, further cause the computing system to generate a recommended list of one or more infections for molecular analysis.

56. The system for infection tracking and monitoring of any one of claims 30-55, wherein the instructions, when executed by the one or more processors, further cause the computing system to identify one or more infections as a priority for analysis and generate or transmit an order for a molecular analysis of the one or more infections.

57. The system for infection tracking and monitoring of any one of claims 30-56, wherein updated patient data, updated healthcare worker data, updated instrument data, and updated infection data are obtained periodically or on demand at least on a daily basis or more frequently than a daily basis; and
wherein the determining of interaction data for each patient occurs on a daily basis or more frequently than a daily basis.
